SEQUENCE LISTING (33 sequences)

SEQ ID NO: 1 - DNA, 20 bp, Artificial Sequence
  Synthetic
ctccgggtaaactcgagata

SEQ ID NO: 2 - DNA, 17 bp, Artificial Sequence
  Synthetic
ctccaggtaaactcgag

SEQ ID NO: 3 - DNA, 17 bp, Artificial Sequence
  Synthetic
acccaggtaccacggtt

SEQ ID NO: 4 - DNA, 25 bp, Artificial Sequence
  Synthetic
gtgacaaacgggtaaacctcgagat

SEQ ID NO: 5 - DNA, 25 bp, Artificial Sequence
  Synthetic
gtgacaaacaggtaaacctcgagat

SEQ ID NO: 6 - DNA, 25 bp, Artificial Sequence
  Synthetic
gtgacaaacaggtaccactcgagat

SEQ ID NO: 7 - DNA, 25 bp, Artificial Sequence
  Synthetic
gtgacaaacaggtaaatctcgagat

SEQ ID NO: 8 - DNA, 20 bp, Artificial Sequence
  Synthetic
aaacaggtaaatctcgagat

SEQ ID NO: 9 - DNA, 34 bp, Artificial Sequence
  Synthetic
ataacttcgtatagcatacattatacgaagttat

SEQ ID NO: 10 - DNA, 22 bp, Artificial Sequence
  Synthetic
gccaccatggagtttgggctga

SEQ ID NO: 11 - DNA, 20 bp, Artificial Sequence
  Synthetic
ctattactaaacacagcatg

SEQ ID NO: 12 - DNA, 34 bp, Artificial Sequence; identical to SEQ ID NO: 9
  Synthetic

SEQ ID NO: 13 - DNA, 29 bp, Artificial Sequence
  Synthetic
tatgaagcttggatatactaagcttcata

SEQ ID NO: 14 - DNA, 34 bp, Artificial Sequence
  Synthetic
atcacctcatacagcatacattatacaaagttat

SEQ ID NO: 15 - protein, 9 aa, Artificial Sequence
  Synthetic
HHHHHHHHH

SEQ ID NO: 16 - protein, 9 aa, Artificial Sequence
  Synthetic
DYKDDDDKG

SEQ ID NO: 17 - DNA, 9 bp, Artificial Sequence
  Synthetic
cttgtgaca

SEQ ID NO: 18 - DNA, 9 bp, Artificial Sequence
  Synthetic
caggtaaat

SEQ ID NO: 19 - DNA, 1940 bp, Artificial Sequence
  Synthetic
atggaattggggctgagctgggttttccttgttgctattttagaaggtgtccagtgtgaggtgcagttggtggagtctgggggaggcttggtccagcctggggggtccctgagactctcctgtgcagcctctggattcacctttagtaaccattggatgaactgggtccgccagactccagggaaagggctggagtgggtggccgccataaacgaagatggaagtgagaaatactatgtgggctctgtgaaggtccgattcaccatctccagagacaacgccaagaactcactgtatctgcaaatgaacagcctgagagtcgaggacacggctgtgtattactgtgcaagagattactatgatatacttacagattactacatacactactggtacttcgatctctggggccgtggcaccctggtcactgtctcctcagcctccaccaagggcccatcggtcttcccgctagcaccctcctccaagagcacctctgggggcacagcggccctgggctgcctggtcaaggactacttccccgaacctgtgacggtgtcgtggaactcaggcgccctgaccagcggcgtgcacaccttcccggctgtcctacagtcctcaggactctactccctcagcagcgtggtgacagtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaggaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaactcgagataacttcgtatagcatacattatacgaagttatctgagcctcctccatccactgtctccaacatggcgaccgttgctgttctggttgtccttggagctgcaatagtcactggagctgtggtggcttttgtgatgaagatgagaaggagaaacacaggtggaaaaggaggggactatgctctggctccaggctcccagacctctgatctgtctctcccagattgtaaagtgatggttcatgaccctcattctctagcgtgaagatcttgaataacttcgtatagcatacattatacgaagttattggcctaggcgcgccgcaggtcgcggccgcgactctagaggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactga

SEQ ID NO: 20 - protein, 564 aa, Artificial Sequence [sequence omitted]

SEQ ID NO: 21 - protein, 480 aa, Artificial Sequence
  Synthetic
MELGLSWVFLVAILEGVQCEVQLVESGGGLVQPGGSLRLSCAASGFTFSNHWMNWVRQTPGKGLEWVAAINEDGSEKYYVGSVKVRFTISRDNAKNSLYLQMNSLRVEDTAVYYCARDYYDILTDYYIHYWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPDSNLWN

SEQ ID NO: 22 - DNA, 1943 bp, Artificial Sequence
  Synthetic
atggaattggggctgagctgggttttccttgttgctattttagaaggtgtccagtgtgaggtgcagttggtggagtctgggggaggcttggtccagcctggggggtccctgagactctcctgtgcagcctctggattcacctttagtaaccattggatgaactgggtccgccagactccagggaaagggctggagtgggtggccgccataaacgaagatggaagtgagaaatactatgtgggctctgtgaaggtccgattcaccatctccagagacaacgccaagaactcactgtatctgcaaatgaacagcctgagagtcgaggacacggctgtgtattactgtgtgagagattactatgatatacttacagattactacatacactactggtacttcgatctctggggccgtggcaccctggtcactgtctcctcagcctccaccaagggcccatcggtcttcccgctagcaccctcctccaagagcacctctgggggcacagcggccctgggctgcctggtcaaggactacttccccgaacctgtgacggtgtcgtggaactcaggcgccctgaccagcggcgtgcacaccttcccggctgtcctacagtcctcaggactctactccctcagcagcgtggtgacagtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaggaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaactcgagataacttcgtatagcatacattatacgaagttatctgagcctcctccatccactgtctccaacatggcgaccgttgctgttctggttgtccttggagctgcaatagtcactggagctgtggtggcttttgtgatgaagatgagaaggagaaacacaggtggaaaaggaggggactatgctctggctccaggctcccagacctctgatctgtctctcccagattgtaaagtgatggttcatgaccctcattctctagcgtgaagatcttgaataacttcgtatagcatacattatacgaagttattggcctaggcgcgccgcaggtcgcggccgcgactctagaggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatga

SEQ ID NO: 23 - protein, 564 aa, Artificial Sequence
  Synthetic
MELGLSWVFLVAILEGVQCEVQLVESGGGLVQPGGSLRLSCAASGFTFSNHWMNWVRQTPGKGLEWVAAINEDGSEKYYVGSVKVRFTISRDNAKNSLYLQMNSLRVEDTAVYYCVRDYYDILTDYYIHYWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKLEITSYSIHYTKLSEPPPSTVSNMATVAVLVVLGAAIVTGAVVAFVMKMRRRNTGGKGGDYALAPGSQTSDLSLPDCKVMVHDPHSLA

SEQ ID NO: 24 - protein, 480 aa, Artificial Sequence
  Synthetic
MELGLSWVFLVAILEGVQCEVQLVESGGGLVQPGGSLRLSCAASGFTFSNHWMNWVRQTPGKGLEWVAAINEDGSEKYYVGSVKVRFTISRDNAKNSLYLQMNSLRVEDTAVYYCVRDYYDILTDYYIHYWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPDSNLWN

SEQ ID NO: 25 - DNA, 3044 bp, Artificial Sequence
  Synthetic
atggaattggggctgagctgggttttccttgttgctattttagaaggtgtccagtgtgaggtgcagttggtggagtctgggggaggcttggtccagcctggggggtccctgagactctcctgtgcagcctctggattcacctttagtaaccattggatgaactgggtccgccagactccagggaaagggctggagtgggtggccgccataaacgaagatggaagtgagaaatactatgtgggctctgtgaaggtccgattcaccatctccagagacaacgccaagaactcactgtatctgcaaatgaacagcctgagagtcgaggacacggctgtgtattactgtgcaagagattactatgatatacttacagattactacatacactactggtacttcgatctctggggccgtggcaccctggtcactgtctcctcagcctccaccaagggcccatcggtcttcccgctagcaccctcctccaagagcacctctgggggcacagcggccctgggctgcctggtcaaggactacttccccgaaccagtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaacaggtaaatctcgagataacttcgtatagcatacattatacgaagttattgggcggtagtggaggttctggtgtgagcaagggcgaggagaccacaatgggcgtaatcaagcccgacatgaagatcaagctgaagatggagggcaacgtgaatggccacgccttcgtgatcgagggcgagggcgagggcaagccctacgacggcaccaacaccatcaacctggaggtgaaggagggagcccccctgcccttctcctacgacattctgaccaccgcgttcagttacggcaacagggccttcaccaagtaccccgacgacatccccaactacttcaagcagtccttccccgagggctactcttgggagcgcaccatgaccttcgaggacaagggcatcgtgaaggtgaagtccgacatctccatggaggaggactccttcatctacgagatacacctcaagggcgagaacttcccccccaacggccccgtgatgcagaaggagaccaccggctgggacgcctccaccgagaggatgtacgtgcgcgacggcgtgctgaagggcgacgtcaagatgaagctgctgctggagggcggcggccaccaccgcgttgacttcaagaccatctacagggccaagaaggcggtgaagctgcccgactatcactttgtggaccaccgcatcgagatcctgaaccacgacaaggactacaacaaggtgaccgtttacgagatcgccgtggcccgcaactccaccgacggcatggacgagctgtacaagtgatggcctaggcgcgccgcaggtcgcggccgcgactctagaggatctttgtgaaggaaccttacttctgtggtgtgacataattggacaaactacctacagagatttaaagctctaaggtaaatataaaatttttaagtgtataatgtgttaaactactgattctaattgtttgtgtattttaggattccaacggcggcggtgcttcaggagctggtagtggcagtggaggtgcatctggtgtaagcaagggtgaggaaaacaacatggcaattataaaggagtttatgaggttcaaggtacacatggaaggttctgtaaatgggcatgaatttgaaatagaaggcgaaggcgagggtagaccttacgaggcatttcaaacagctaagctgaaggttactaaaggcgggcctctgccttttgcctgggatatactttcaccacagtttatgtatggcagtaaagtgtatataaagcatccagctgatatacccgactactttaagttgagctttccggagggctttaagtgggagagagttatgaacttcgaggacggcggtataatacatgttaaccaagacagcagcctacaagatggtgtgttcatttacaaggttaagctgaggggtaccaacttccctagcgacggccctgtaatgcagaagaaaacaatgggttgggaagccagcagcgaacggatgtacccagaggacggtgctttaaaatctgaaattaagaagcggttaaagctgaaagatggagggcactatgcaagtgaagtaaaaactacttacaaagcaaagaaacccgtacaactacccggtgcatacattgttgatatcaaacttgacattaccagccataacgaggactacacaatagtagaacagtatgaacgtgctgagggcaggcacagtactggtggtatggacgaactgtataagtacactgatatcgaaatgaaccgcctgggtaagtgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtct

SEQ ID NO: 26 - protein, 510 aa, Artificial Sequence
  Synthetic
MELGLSWVFLVAILEGVQCEVQLVESGGGLVQPGGSLRLSCAASGFTFSNHWMNWVRQTPGKGLEWVAAINEDGSEKYYVGSVKVRFTISRDNAKNSLYLQMNSLRVEDTAVYYCARDYYDILTDYYIHYWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKQVNLEITSYSIHYTKLLGGSGGSGVSKGEETTMGVIKPDMKIKLKMEGNVNGHAFVIEGEGEGKPYDGTNTINLEVKEGAPLPFSYDILTTAFSYGNRAFTKYPDDIPNYFKQSFPEGYSWERTMTFEDKGIVKVKSDISMEEDSFIYEIHLKGENFPPNGPVMQKETTGWDASTERMYVRDGVLKGDVKMKLLLEGGGHHRVDFKTIYRAKKAVKLPDYHFVDHRIEILNHDKDYNKVTVYEIAVARNSTDGMDELYK

SEQ ID NO: 27 - DNA, 2085 bp, Artificial Sequence
  Synthetic
atggaattggggctgagctgggttttccttgttgctattttagaaggtgtccagtgtgaggtgcagttggtggagtctgggggaggcttggtccagcctggggggtccctgagactctcctgtgcagcctctggattcacctttagtaaccattggatgaactgggtccgccagactccagggaaagggctggagtgggtggccgccataaacgaagatggaagtgagaaatactatgtgggctctgtgaaggtccgattcaccatctccagagacaacgccaagaactcactgtatctgcaaatgaacagcctgagagtcgaggacacggctgtgtattactgtgcaagagattactatgatatacttacagattactacatacactactggtacttcgatctctggggccgtggcaccctggtcactgtctcctcagcctccaccaagggcccatcggtcttcccgctagcaccctcctccaagagcacctctgggggcacagcggccctgggctgcctggtcaaggactacttccccgaaccagtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaacaggattccaacggcggcggtgcttcaggagctggtagtggcagtggaggtgcatctggtgtaagcaagggtgaggaaaacaacatggcaattataaaggagtttatgaggttcaaggtacacatggaaggttctgtaaatgggcatgaatttgaaatagaaggcgaaggcgagggtagaccttacgaggcatttcaaacagctaagctgaaggttactaaaggcgggcctctgccttttgcctgggatatactttcaccacagtttatgtatggcagtaaagtgtatataaagcatccagctgatatacccgactactttaagttgagctttccggagggctttaagtgggagagagttatgaacttcgaggacggcggtataatacatgttaaccaagacagcagcctacaagatggtgtgttcatttacaaggttaagctgaggggtaccaacttccctagcgacggccctgtaatgcagaagaaaacaatgggttgggaagccagcagcgaacggatgtacccagaggacggtgctttaaaatctgaaattaagaagcggttaaagctgaaagatggagggcactatgcaagtgaagtaaaaactacttacaaagcaaagaaacccgtacaactacccggtgcatacattgttgatatcaaacttgacattaccagccataacgaggactacacaatagtagaacagtatgaacgtgctgagggcaggcacagtactggtggtatggacgaactgtataagtacactgatatcgaaatgaaccgcctgggtaagtgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtct

SEQ ID NO: 28 - protein, 517 aa, Artificial Sequence
  Synthetic
MELGLSWVFLVAILEGVQCEVQLVESGGGLVQPGGSLRLSCAASGFTFSNHWMNWVRQTPGKGLEWVAAINEDGSEKYYVGSVKVRFTISRDNAKNSLYLQMNSLRVEDTAVYYCARDYYDILTDYYIHYWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKQDSNGGGASGAGSGSGGASGVSKGEENNMAIIKEFMRFKVHMEGSVNGHEFEIEGEGEGRPYEAFQTAKLKVTKGGPLPFAWDILSPQFMYGSKVYIKHPADIPDYFKLSFPEGFKWERVMNFEDGGIIHVNQDSSLQDGVFIYKVKLRGTNFPSDGPVMQKKTMGWEASSERMYPEDGALKSEIKKRLKLKDGGHYASEVKTTYKAKKPVQLPGAYIVDIKLDITSHNEDYTIVEQYERAEGRHSTGGMDELYKYTDIEMNRLGK

SEQ ID NO: 29 - DNA, 2588 bp, Artificial Sequence
  Synthetic
atgagggcctggatcttctttctcctttgcctggccgggagggccttggcagaggttcagctggtggagtctggcggtggcctggtgcagcccgggggctctctccgtttgtcctgtgcagcttctggcttcaacattaaagacacctatatccactgggtgcgtcaggctccgggtaagggcctggagtgggttgcaaggatttatcctacgaatggttatactcgttatgccgatagcgtcaagggccgtttcactataagcgcagacacttcgaaaaacacagcctacctccagatgaacagcctgcgtgctgaggacactgccgtctattattgtagcagatggggtggggacggcttctatgctatggactactggggtcaaggtacactagtcaccgtcagcagcgcttccaccaagggcccatccgtcttcccgctagcaccctcctccaagagcacctctgggggcacagcggccctgggctgcctggtcaaggactacttccccgaaccagtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaacaggtaaatctcgagataacttcgtatagcatacattatacgaagttatctgagcctcctccatccactgtctccaacatggcgaccgttgctgttctggttgtccttggagctgcaatagtcactggagctgtggtggcttttgtgatgaagatgagaaggagaaacacaggaggaaaaggaggggactatgctctggctccaggctcccagacctctgatctgtctctcccagattgtaaagtgatggttcatgaccctcattctctagcgtgaagatcttgatggcctaggcgcgccgcaggtcgcggccgcgactctagaggatctttgtgaaggaaccttacttctgtggtgtgacataattggacaaactacctacagagatttaaagctctaaggtaaatataaaatttttaagtgtataatgtgttaaactactgattctaattgtttgtgtattttaggattccaacggcggcggtgcttcaggagctggtagtggcagtggaggtgcatctggtgtcacatcaatcacattagatctagtaaatccgactgccggtcaatactcatctttcgtggataaaatccgaaacaacgtaaaggatccaaacctgaaatacggtggtaccgacatagccgtgataggcccaccttctaaagataaattccttagaattaatttccaaagttcccgaggaacggtctcacttggcctaaaacgcgataacttgtacgtggtcgcgtatcttgcaatggataacacgaatgttaatcgggcatattacttcaaatcagaaattacttccgccgagttaaccgcccttttcccagaggccacaactgcaaatcagaaagctttagaatacacagaagattatcagtcgatcgaaaagaatgcccagataacacagggagataaaagtagaaaagaactcgggttggggatcgacttacttttgacgttcatggaagcagtgaacaagaaggcacgtgtggttaaaaacgaagctaggtttctgcttatcgctattcaaatgacagctgaggtagcacgatttaggtacattcaaaacttggtaactaagaacttccccaacaagttcgactcggataacaaggtgattcaatttgaagtcagctggcgtaagatttctacggcaatatacggggatgccaaaaacggcgtgtttaataaagattatgatttcgggtttggaaaagtgaggcaggtgaaggacttgcaaatgggactccttatgtatttgggcaaaccaaagtcgtcaaacgaggcgaattctactgcttacgccactacggtcctctgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtct

SEQ ID NO: 30 - protein, 333 aa, Artificial Sequence
  Synthetic
MRAWIFFLLCLAGRALAEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKQVNLEITSYSIHYTKLSEPPPSTVSNMATVAVLVVLGAAIVTGAVVAFVMKMRRRNTGGKGGDYALAPGSQTSDLSLPDCKVMVHDPHSLA

SEQ ID NO: 31 - DNA, 2124 bp, Artificial Sequence
  Synthetic
atgagggcctggatcttctttctcctttgcctggccgggagggccttggcagaggttcagctggtggagtctggcggtggcctggtgcagcccgggggctctctccgtttgtcctgtgcagcttctggcttcaacattaaagacacctatatccactgggtgcgtcaggctccgggtaagggcctggagtgggttgcaaggatttatcctacgaatggttatactcgttatgccgatagcgtcaagggccgtttcactataagcgcagacacttcgaaaaacacagcctacctccagatgaacagcctgcgtgctgaggacactgccgtctattattgtagcagatggggtggggacggcttctatgctatggactactggggtcaaggtacactagtcaccgtcagcagcgcttccaccaagggcccatccgtcttcccgctagcaccctcctccaagagcacctctgggggcacagcggccctgggctgcctggtcaaggactacttccccgaaccagtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaacaggattccaacggcggcggtgcttcaggagctggtagtggcagtggaggtgcatctggtgtcacatcaatcacattagatctagtaaatccgactgccggtcaatactcatctttcgtggataaaatccgaaacaacgtaaaggatccaaacctgaaatacggtggtaccgacatagccgtgataggcccaccttctaaagataaattccttagaattaatttccaaagttcccgaggaacggtctcacttggcctaaaacgcgataacttgtacgtggtcgcgtatcttgcaatggataacacgaatgttaatcgggcatattacttcaaatcagaaattacttccgccgagttaaccgcccttttcccagaggccacaactgcaaatcagaaagctttagaatacacagaagattatcagtcgatcgaaaagaatgcccagataacacagggagataaaagtagaaaagaactcgggttggggatcgacttacttttgacgttcatggaagcagtgaacaagaaggcacgtgtggttaaaaacgaagctaggtttctgcttatcgctattcaaatgacagctgaggtagcacgatttaggtacattcaaaacttggtaactaagaacttccccaacaagttcgactcggataacaaggtgattcaatttgaagtcagctggcgtaagatttctacggcaatatacggggatgccaaaaacggcgtgtttaataaagattatgatttcgggtttggaaaagtgaggcaggtgaaggacttgcaaatgggactccttatgtatttgggcaaaccaaagtcgtcaaacgaggcgaattctactgcttacgccactacggtcctctgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtct

SEQ ID NO: 32 - protein, 530 aa, Artificial Sequence [sequence omitted]

SEQ ID NO: 33 - protein, 181 aa, Artificial Sequence [sequence omitted]

(19) United States
(12) Patent Application Publication
(10) Pub. No.: US 2013/0035472 A1
(43) Pub. Date: Feb. 7, 2013

(54) METHOD OF PRODUCING TRANSCRIPTS USING CRYPTIC SPLICE SITES

(71) Applicants: Robert Horlick, San Diego, CA (US); John Macomber, San Diego, CA (US); Andrew Cubitt, San Diego, CA (US); David King, Encinitas, CA (US)

(72) Inventors: Robert Horlick, San Diego, CA (US); John Macomber, San Diego, CA (US); Andrew Cubitt, San Diego, CA (US); David King, Encinitas, CA (US)

(73) Assignee: ANAPTYSBIO, INC., San Diego, CA (US)

(21) Appl. No.: 13/634,279

(22) PCT Filed: Mar. 15, 2011
(86) PCT No.: PCT/US11/28529

§ 371 Date: Oct. 23, 2012

Related U.S. Application Data
(60) Provisional application No. 61/314,811, filed on Mar. 17, 2010.

(51) Int. Cl. C12N 15/11; C12P 19/34; C07K 14/00; C12N 5/10; C12N 15/63; C12N 1/21; C12N 1/19; C07H 1/00; C07K 19/00

(52) U.S. Cl. 530/350; 536/25.3; 536/23.1; 435/91.3; 435/320.1; 435/252.31; 435/252.33; 435/252.34; 435/252.35; 435/254.2; 435/348; 435/325; 435/254.23; 435/254.21; 435/358; 435/369; 435/357; 435/354; 435/367; 435/352; 435/366; 435/252.3

(57) ABSTRACT

The invention is directed to a method of preparing a nucleic acid sequence with a modified splice site usage profile, which employs the use of a nucleic acid sequence comprising a cryptic splice donor site. The invention also provides a method of producing an alternate form of an RNA molecule encoded by a nucleic acid sequence, which nucleic acid sequence comprises a cryptic splice donor site, a heterologous nucleic acid sequence, and a splice acceptor site.

### CROSS-REFERENCE TO RELATED APPLICATIONS

This patent application claims the benefit of U.S. Provisional Patent Application No. 61/314,811, filed Mar. 17, 2010, which is incorporated by reference.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 58,967 Byte ASCII (Text) file named “707758_ST25.txt,” created on Mar. 15, 2011.

### BACKGROUND OF THE INVENTION

Splicing is a complex process that removes introns and joins exons within an RNA transcript. The intron-exon junctions within an RNA transcript are known as splice sites, which are recognized by specialized RNA and protein subunits known as the spliceosome. The 5′ junction of an intron is known as the splice donor site, while the 3′ end of an intron is referred to as the splice acceptor site. Splice donors are generally identified by homology to various known consensus sequences, most of which are characterized by a canonical GT motif at the beginning of the splice donor site (see Mount, Nucleic Acid Res., 10: 459-472 (1982)).

The presence of multiple splice donors within an RNA transcript may lead to the expression of multiple proteins from the same RNA transcript. Known as alternative splicing, different splice donors within an RNA transcript may be reconnected in multiple ways with a downstream splice acceptor to generate different mRNAs, each of which may be translated into a different protein isoform. As a result, alternative splicing is a useful means of encoding multiple proteins within a single gene.

Alternative splicing offers various practical applications in the synthesis and expression of proteins. For example, alternative splicing of a transmembrane protein may be utilized to remove its membrane-spanning domain such that the protein is secreted when expressed. In addition, alternative splicing of an RNA transcript may produce two different protein isoforms, one of which may be covalently linked to another moiety that permits facile detection (as in the case of a fluorescent label), purification (as in the case of a poly-histidine tag), or mediates cell killing (e.g., when conjugated to a cytotoxic agent), while the other isoform does not contain such a covalently bound protein.

However, the aforementioned applications of alternative splicing can only be utilized in RNA transcripts in which multiple splice donor sites are present. If an RNA transcript does not have at least two such splice donor sites, it is generally difficult to generate multiple expressed proteins from a single gene transcript. Thus, there remains a need for improved methods for producing proteins in eukaryotic cells, including methods for producing alternate forms of the same protein. This invention provides such methods.

### BRIEF SUMMARY OF THE INVENTION

The invention provides a method of preparing a nucleic acid sequence with a modified splice site usage profile. The method comprises (a) providing a nucleic acid sequence encoding a gene product of interest, wherein the nucleic acid sequence comprises a cryptic splice donor site and a splice acceptor site; and (b) mutating the nucleic acid sequence to provide a mutant nucleic acid sequence that has a splice site usage profile that differs from the splice site usage profile of the nucleic acid sequence prior to mutation.

The invention also provides an isolated nucleic acid sequence encoding a gene product of interest. The nucleic acid sequence comprises (a) a cryptic splice donor site, (b) a heterologous nucleic acid sequence, and (c) a splice acceptor site, wherein at least two different transcripts are produced when the nucleic acid sequence is introduced into a cell.

Also provided by the invention is a method of producing an alternate form of an RNA molecule encoded by a nucleic acid sequence. The method comprises (a) preparing a nucleic acid sequence encoding an RNA molecule, wherein the nucleic acid sequence comprises (i) a cryptic splice donor site, (ii) a heterologous nucleic acid sequence, and (iii) a splice acceptor site, and (b) introducing the nucleic acid sequence into a host cell, such that RNA splicing occurs between the cryptic splice donor site and the splice acceptor site to produce an alternate form of the RNA molecule.

### BRIEF DESCRIPTION OF THE SEVERAL VIEWS OF THE DRAWING(S)

FIGS. 1A-1K are diagrams depicting mechanisms by which antibody heavy chain-encoding nucleic acid sequences can be alternately spliced to generate membrane-bound or secreted full-length antibodies, or fragments thereof, by utilizing cryptic splice sites. Splice donors are shown as diagonal lines, and splice acceptors are shown in black. V_{H} denotes the variable region. C_{H1}, C_{H2}, and C_{H3} denote constant domains. “SC” denotes a stop codon.

FIG. 1A is a diagram depicting the native structure of a nucleic acid sequence encoding a typical antibody heavy chain comprising a transmembrane domain. FIG. 1B is a diagram depicting a nucleic acid sequence which can be spliced to generate a secreted full-length antibody and full length membrane-bound antibody. FIG. 1C is a diagram depicting a nucleic acid sequence which can be spliced to generate a secreted Facb fragment and a membrane-bound full-length antibody. FIG. 1D is a diagram depicting a nucleic acid sequence which can be spliced to generate a membrane-bound Facb and membrane-bound full-length antibody. FIG. 1E is a diagram depicting a nucleic acid sequence which can be spliced to generate a membrane-bound Facb fragment and a secreted full-length antibody.

FIG. 1F is a diagram depicting a nucleic acid sequence which can be spliced to generate a membrane-bound Facb fragment and a secreted Facb fragment. FIG. 1G is a diagram depicting a nucleic acid sequence which can be spliced to generate a secreted Fab fragment and a membrane-bound full-length antibody. FIG. 1H is a diagram depicting a nucleic acid sequence which can be spliced to generate a membrane-bound Fab and membrane-bound full-length antibody. FIG. 1I is a diagram depicting a nucleic acid sequence which can be spliced to generate a membrane-bound Fab and secreted full-length antibody. FIG. 1J is a diagram depicting a nucleic acid sequence which can be spliced to generate a secreted Fab fragment and membrane-bound full-length antibody. FIG. 1K is a diagram depicting a nucleic acid sequence which can be spliced to generate a secreted Fab fragment and membrane-bound Fab fragment.

FIGS. 2A-2F are diagrams depicting nucleic acid sequence constructs T1-T6, each of which comprises a cryptic splice donor site and a mutation which alters the splice site usage profile of the nucleic acid sequence. Specifically, a H2kk peritransmembrane (H2kk), transmembrane (tm) and cytoplasmic domain (CD) were appended to the human IgG1 heavy chain constant region (not including the stop codon) to generate chimeric immunoglobulin genes. In FIG. 2E, “*” denotes a 36-nucleotide deletion, and in FIG. 2F, “tag” denotes an insertion of a FLAG or H is fusion domain to the 3′ splice acceptor. “SA” denotes a splice acceptor, and “SD” denotes a splice donor.

FIG. 3 is an image of a gel which illustrates the sizes of various DNA fragments generated by amplification of the mRNA expressed in HEK293 from the T1 and T2 genes. The DNA fragments that were generated due to unmasking of various different splice donor sites (including SD4, SD3 and SD2) in the T2 gene sequence are indicated by arrows.

FIG. 4 is an image of a gel which illustrates the sizes of various DNA fragments generated by amplification of the mRNA expressed in HEK293 from the T1, T2, and T5 genes. The DNA fragments that were generated due to unmasking of various different splice donor sites (including SD4, SD3 and SD2) in the T2 gene sequence are indicated by arrows.

FIGS. 5A-5C are images of a Western blot in which the polypeptide encoded by the T6 construct is stained with an anti-Fc antibody (FIG. 5A), an anti-His antibody (FIG. 5B), and an anti-FLAG antibody (FIG. 5C).

FIGS. 6A-6F are diagrams depicting the nucleic acid sequence constructs described in Example 6, each of which comprises a cryptic splice donor site and a mutation which alters the splice site usage profile of the nucleic acid sequence. A H2kk peritransmembrane, transmembrane (tm), and cytoplasmic domain were appended to the variable (IgHV) and Fab constant regions of a human IgG1 heavy chain polypeptide to generate chimeric immunoglobulin genes. One or more Loxp sites were inserted on either side of the H2kk transmembrane domain. “SA” denotes a splice acceptor, and “SD” denotes a splice donor.

FIG. 7 is a graph which illustrates the average number of surface Fab molecules per cell. The Fab molecules are produced by the nucleic acid constructs described in Example 6.

FIG. 8 is a graph which compares the potency of three control anti-IL-17 antibodies as compared to the anti-IL-17 antibodies generated as described in Example 7, as measured by an HTRF assay.

FIG. 9 is a graph which illustrates the ability of the anti-IL-17 antibodies described in Example 7 to inhibit IL-6 release in NIH3T3 cells.

### DETAILED DESCRIPTION OF THE INVENTION

The invention provides a method of preparing a nucleic acid sequence with a modified splice site usage profile. The method comprises (a) providing a nucleic acid sequence encoding a gene product of interest, wherein the nucleic acid sequence comprises a cryptic splice donor site; and (b) mutating the nucleic acid sequence to provide a mutant nucleic acid sequence that has a splice site usage profile that differs from the splice site usage profile of the nucleic acid sequence prior to mutation. The invention also provides an isolated nucleic acid sequence encoding a gene product of interest, which comprises (a) a cryptic splice donor site; (b) a heterologous nucleic acid sequence; and (c) a splice acceptor site; wherein at least two different transcripts are produced when the nucleic acid sequence is introduced into a cell.

By “nucleic acid sequence” is meant a polymer of DNA or RNA, i.e., a polynucleotide, which can be single-stranded or double-stranded and which can contain non-natural or altered nucleotides. Nucleic acids are typically linked via phosphate bonds to form nucleic acids or polynucleotides, though many other linkages are known in the art (e.g., phosphorothioates, boranophosphates, and the like). The nucleic acid sequence can be eukaryotic or prokaryotic in origin. Preferably, the nucleic acid sequence is eukaryotic in origin. In this regard, eukaryotic genes are comprised of “exons” and “introns.” The term “exon,” as used herein, refers to a nucleic acid sequence present in a gene which is represented in the mature form of an RNA molecule after excision of introns during transcription. Exons are translated into protein. The term “intron,” as used herein, refers to a nucleic acid sequence present in a given gene which is not translated into protein and is generally found between exons. During transcription, introns are removed from precursor messenger RNA (pre-mRNA), and exons are joined via RNA splicing. Thus, in a preferred embodiment of the invention, the nucleic acid sequence comprises one or more exons and introns. The term “transcription,” as used herein, is the process of creating an equivalent RNA copy of a sequence of DNA, and involves the steps of initiation, elongation, termination, and RNA processing (which includes splicing) (see, e.g., Griffiths et al., eds., Modern Genetic Analsysis: Integrating Genes and Genomes, 2^{nd} ed., W.H. Freeman and Co., New York (2002)).

RNA splicing is catalyzed by a large RNA-protein complex called the spliceosome, which is comprised of five small nuclear ribonucleoproteins (snRNPs) (see, e.g., Watson et al. (eds.), Molecular Biology of the Gene, 6^{th} Edition, Cold Spring Harbor Laboratory Press, Cold Spring Harbor, N.Y. (2008)). The borders between introns and exons are marked by specific nucleotide sequences within a pre-mRNA, which delineate where splicing will occur. Such boundaries are referred to herein as “splice sites.” The term “splice site,” as used herein, refers to polynucleotides that are capable of being recognized by the spicing machinery of a eukaryotic cell as suitable for being cut and/or ligated to another splice site. Splice sites allow for the excision of introns present in a pre-mRNA transcript. Typically, the 5′ splice boundary is referred to as the “splice donor site” or the “5′ splice site,” and the 3′ splice boundary is referred to as the “splice acceptor site” or the “3′ splice site.” Splice sites include, for example, naturally occurring splice sites, engineered or synthetic splice sites, canonical or consensus splice sites, and/or non-canonical splice sites, for example, cryptic splice sites. In addition to the 5′ and 3′ splice sites, RNA splicing also requires a third sequence called the branch point site. The branch point site typically is located entirely within an intron close to its 3′ end, and is followed by a polypyrimidine tract.

The terms “canonical splice site” or “consensus splice site” can be used interchangeably and refer to splice sites that are conserved across species. Consensus sequences for the 5′ splice site and the 3′ splice site used in eukaryotic RNA splicing are well known in the art (see, e.g., Gesteland et al. (eds.), The RNA World, 3^{rd} Edition, Cold Spring Harbor Laboratory Press, Cold Spring Harbor, N.Y., (2006), Watson et al., supra, and Mount, Nucleic Acid Res., 10: 459-472 (1982)). These consensus sequences include nearly invariant dinucleotides at each end of the intron: GT at the 5′ end of the intron, and AG at the 3′ end of an intron. The splice donor site consensus sequence is (for DNA) AG/GTRAGT (where A is adenosine, T is thymine, G is guanine, C is cytosine, R is a purine and “/” is the splice site). Non-consensus splice donor sites include, for example, SEQ ID NO: 1, SEQ ID NO: 2, SEQ ID NO: 3, SEQ ID NO: 4, SEQ ID NO: 5, SEQ ID NO: 6, SEQ ID NO: 7, and SEQ ID NO: 8. The splice acceptor site consists of three separate sequence elements: the branch point or branch site, a polypyrimidine tract and the 3′ consensus sequence. The branch point consensus sequence in eukaryotes is YNYTRAC (where Y is a pyrimidine, N is any nucleotide, and R is a purine; the underlined A is the site of branch formation. The 3′ splice site consensus sequence is YAG (where Y is a pyrimidine) (see, e.g., Griffiths et al., eds., Modern Genetic Analysis, 2^{nd} edition, W.H. Freeman and Company, New York (2002)). The 3′ splice acceptor site typically is located at the 3′ end of an intron, and, in the context of the invention, can be located within the 3′ untranslated region of the nucleic acid sequence comprising the cryptic splice donor site. Modified consensus sequences that maintain the ability to function as 5′ donor splice sites and 3′ splice acceptor sites may be used in connection with the invention.

The term “cryptic splice donor site,” as used herein, refers to a nucleic acid sequence which does not normally function as a splice donor site, but can be activated to become a functioning splice donor site. In the context of the invention, a cryptic splice donor site preferably comprises a GT sequence. Most preferably, the cryptic splice donor site is at least 50%, at least 55%, at least 60%, at least 65%, at least 70%, at least 75%, at least 80%, at least 85%, at least 90%, at least 95%, at least 100%, or a range defined by any two of the foregoing values, identical to the sequence CAGGTRAGT (where R is A or G).

The nucleic acid sequence comprises at least one cryptic splice donor site, and can comprise multiple cryptic splice donor sites. For example, the nucleic acid sequence can comprise 2-20 (e.g., 2, 3, 5, 10, 15, 20, or ranges thereof) cryptic splice donor sites. In addition, the cryptic splice donor site can be located anywhere in the nucleic acid sequence, so long as its location does not prevent recognition of the cryptic splice donor site by the spliceosome after activation of the cryptic splice donor site (such as by, e.g., mutation of the nucleic acid sequence as described herein). For example, the cryptic splice donor site can be located within an open reading frame (ORF) of the nucleic acid sequence. Alternatively, the cryptic splice donor site can be located within a 5′ untranslated region of the nucleic acid sequence. One of ordinary skill in the art will appreciate that efficiency with which the cryptic splice donor site is activated (such as by, e.g., the mutation of the nucleic acid sequence as described herein) may depend on the location of the cryptic splice donor site within the nucleic acid sequence. For example, splicing efficiency may be maximized when the cryptic splice donor site is located within an ORF as compared to when the cryptic splice donor site is located within a 5′ untranslated region of the nucleic acid sequence, or vice versa. In a preferred embodiment, a cryptic splice donor site is located within about 50 nucleotides (upstream or downstream) of the beginning of an intron.

A cryptic splice donor site can be activated by any modification to the nucleic acid molecule in which it is located, so long as the modification positions the cryptic splice site in a context that is recognized by the splicing machinery (i.e., spliceosome) of a cell. Preferably, the nucleic acid molecule is modified by mutation to activate the cryptic splice donor site. In this respect, the invention comprises mutating the nucleic acid sequence encoding a gene product of interest. A variety of different types of mutations can be introduced into the nucleic acid sequence in order to activate the cryptic splice donor site. For example, a point mutation can be introduced into the nucleic acid sequence. The term “point mutation,” as used herein, refers to any change to a single nucleotide. Point mutations include, for example, deletions, transitions, and transversions, and can be classified as nonsense mutations, missense mutations, or silent mutations. A “nonsense” mutation produces a stop codon. A “missense” mutation produces a codon that encodes a different amino acid. A “silent” mutation produces a codon that encodes either the same amino acid or a different amino acid that does not alter the function of the protein. One or more point mutations can be introduced into the nucleic acid sequence comprising the cryptic splice donor site. For example, the nucleic acid sequence comprising the cryptic splice site can be mutated by introducing two or more (e.g., 2, 5, 10, or more) point mutations therein. A point mutation can be introduced at any location within the nucleic acid sequence comprising the cryptic splice donor site. For example, the point mutation can be introduced within a cryptic splice donor site itself. Alternatively, the point mutation can be introduced adjacent to a cryptic splice donor site. For example, the point mutation can be introduced upstream or downstream of a cryptic splice site. In embodiments where the nucleic acid sequence comprising a cryptic splice donor site is mutated by introducing multiple point mutations therein, the point mutations can be introduced upstream and/or downstream of the cryptic splice donor site. In addition, the multiple point mutations can be introduced into the 5′ or 3′ untranslated regions of the nucleic acid sequence comprising the cryptic splice donor site. Alternatively, the multiple point mutations can be introduced directly into the cryptic splice donor site. One of ordinary skill in the art will appreciate that such mutations shift the reading frame of the nucleic acid sequence, and thereby position the cryptic splice donor site in a context that is recognized by the splicing machinery.

In another embodiment of the invention, mutating the nucleic acid sequence encoding a gene product of interest comprises deleting one or more nucleotides of the nucleic acid sequence. The deletion can be of any suitable size, so long as the deletion produces a mutant nucleic acid sequence that has a splice site usage profile that differs from the spice site usage profile of the nucleic acid sequence prior to mutation. Desirably, the deletion comprises at least about 2-1,000 nucleotides. In this respect, the deletion comprises at least about 2 nucleotides, at least about 5 nucleotides, at least about 10 nucleotides, at least about 20 nucleotides, at least about 50 nucleotides, at least about 75 nucleotides, at least about 100 nucleotides, at least about 150 nucleotides, at least about 200 nucleotides, at least about 250 nucleotides, at least about 300 nucleotides, at least about 350 nucleotides, at least about 400 nucleotides, at least about 450 nucleotides, at least about 500 nucleotides, at least about 750 nucleotides, at least about 1,000 nucleotides, or any range therein (e.g., 2-1,000 nucleotides, 10-500 nucleotides, or 50-200 nucleotides).

In a preferred embodiment of the invention, the nucleic acid sequence is mutated by inserting a heterologous nucleic acid sequence therein. By “heterologous nucleic acid sequence” is meant a nucleic acid sequence that is different from the nucleic acid sequence which comprises a cryptic splice donor site. In one embodiment, the heterologous nucleic acid sequence is not obtained or derived from the nucleic acid sequence which comprises a cryptic splice donor site. Alternatively, the heterologous nucleic acid sequence lacks a cryptic splice donor site, but is otherwise identical to the nucleic acid sequence described herein.

The heterologous nucleic acid sequence can be of any suitable size, so long as insertion of the heterologous nucleic acid sequence into the nucleic acid sequence comprising a cryptic splice donor site produces a mutant nucleic acid sequence that has a splice site usage profile that differs from the spice site usage profile of the nucleic acid sequence prior to mutation. Desirably, the heterologous nucleic acid sequence comprises at least about 2-1,000 nucleotides. In this respect, the heterologous nucleic acid sequence comprises at least about 2 nucleotides, at least about 5 nucleotides, at least about 10 nucleotides, at least about 20 nucleotides, at least about 50 nucleotides, at least about 75 nucleotides, at least about 100 nucleotides, at least about 150 nucleotides, at least about 200 nucleotides, at least about 250 nucleotides, at least about 300 nucleotides, at least about 350 nucleotides, at least about 400 nucleotides, at least about 450 nucleotides, at least about 500 nucleotides, at least about 750 nucleotides, at least about 1,000 nucleotides, or any range therein (e.g., 2-1,000 nucleotides, 10-500 nucleotides, or 50-200 nucleotides).

Whatever type of mutation is introduced into the nucleic acid sequence, the mutation preferably induces the formation of a stem-loop structure. For example, the heterologous nucleic acid sequence preferably forms a stem-loop structure by virtue of containing at least one pair of nucleic acids that can form hydrogen bonds within or outside the heterologous nucleic acid sequence. When the mutation is a point mutation (e.g., a deletion), a stem-loop structure forms by way of hydrogen bonding between one or more nucleic acid sequences in the vicinity of the mutation. The term “stem-loop structure,” as used herein, refers to a pattern of intramolecular nucleic acid base pairing that can occur in single-stranded DNA or, more commonly, in RNA, and is also referred to in the art as a “hairpin” or “hairpin loop.” Stem-loop structures are formed when two complementary sequences within the same nucleic acid molecule (which are usually palindromic) base-pair to form a double helix, and the intervening unpaired sequence is looped out. It will be appreciated that the formation of a stem-loop structure is dependent on the stability of the resulting helix and loop regions. The stability of the double helix is determined by its length, the number of mismatches or bulges it contains, and the nucleotide composition of the paired region. Regarding the nucleotide composition of the double helix, pairings between guanine and cytosine have three hydrogen bonds and are more stable compared to adenine-uracil pairings, which have only two hydrogen bonds. For RNA, adenine-uracil pairings featuring two hydrogen bonds are common and favorable to the generation of stem-loop structures. Base stacking interactions also promote helix formation.

Thus, the double helix can comprise about 50 base pairs, about 45 base pairs, about 40 base pairs, about 35 base pairs, about 30 base pairs, about 25 base pairs, about 20 base pairs, about 15 base pairs, about 10 base pairs, about 5 base pairs, about 3 base pairs, or a range defined by any of two of the foregoing values. In a preferred embodiment, the double helix of the stem-loop structure comprises no more than about 50 (e.g., about 50, 45, 40, 35, 30, 25, 20, 15, 10, or 5) base pairs. In addition, the double helix can comprise at least about 3 base pairs. For example, the double helix can comprise at least about 3 base pairs, at least about 5 base pairs, at least about 7 base pairs, or at least about 10 base pairs. Preferably, the double helix (or “stem”) comprises between about 3 and 50 base pairs, between about 5 and 40 base pairs, between about 10 and 30 base pairs, or between about 15 and 25 base pairs. More preferably, the double helix comprises between about 3 and 20 base pairs, between about 4 and 8 base pairs, between about 5 and 15 base pairs, or between about 7 and 12 base pairs.

With respect to the size of the “loop” of the stem-loop structure, loops containing less than three nucleotides are sterically prohibitive and generally do not form. Large loops with no secondary structure of their own (such as pseudoknots) also are unstable. Thus, the loop preferably comprises about 3 to about 50 (e.g., about 3, 5, 7, 10, 15, 20, 25, 30, 35, 40, 45, 50, or ranges thereof) nucleotides. Preferably, the loop comprises between about 3 and 20 nucleotides, between about 4 and 8 nucleotides, between about 5 and 15 nucleotides, or between about 7 and 12 nucleotides. For optimal stability, most preferably the loop comprises between about 4 and 8 nucleotides. Loops comprising the sequence UUCG are known as “tetraloops” and are particularly stable due to the base-stacking interactions of its component nucleotides. The loop structures may or may not be symmetrical in complimentarity with respect to the nucleic acids within the loop, but at least one pair of nucleic acids forms hydrogen bonds within the loop structure. Stem-loop structures are described in greater detail in, e.g., Watson et al., eds., Molecular Biology of the Gene, 6th ed., Cold Spring Harbor Laboratory Press, New York (2008), and Bevilacqua et al., Annu. Rev. Phys. Chem., 59: 79-103 (2008).

The heterologous nucleic acid sequence preferably forms at least one stem-loop structure. However, the heterologous nucleic acid sequence can form multiple stem-loop structures, so long as the nucleic acid sequence comprising a cryptic splice donor site has a splice site usage profile that differs from the splice site usage profile of the nucleic acid sequence prior to insertion of the heterologous nucleic acid sequence. For example, the heterologous nucleic acid sequence can form about 2 to about 20 (e.g., about 2, 3, 4, 5, 6, 7, 8, 9, 10, 15, 20, or ranges thereof) stem-loop structures. Desirably, the heterologous nucleic acid sequence forms between about 2 and about 20 (e.g., about 2, 5, 10, 15, 20, or ranges thereof) stem-loop structures. Preferably, the heterologous nucleic acid sequence forms between about 2 and 15 (e.g., about 2, 5, 8, 10, 12, 15, or ranges thereof) stem-loop structures. More preferably, the heterologous nucleic acid sequence forms between about 2 and 10 (e.g., about 2, 3, 4, 5, 6, 7, 8, 9, 10, or ranges thereof) stem-loop structures. Most preferably, the heterologous nucleic acid sequence forms between about 2 and 5 (e.g., 2, 3, 4, 5, or ranges thereof) stem-loop structures.

In one embodiment, the heterologous nucleic acid sequence comprises a loxp recombination site. Loxp recombination sites typically are used in the art in combination with Cre recombinase to induce site-specific recombination events. Such “Cre-Lox” systems are disclosed in, e.g., Abremski et al., Cell, 32: 1301-1311 (1983), and U.S. Pat. No. 4,959,317. In general, loxp recombination sites comprise an asymmetric sequence of 8 nucleotides flanked on both sides by a palindromic sequence of 13 nucleotides. Preferably, the loxp recombination site comprises the sequence ATAACTTCGTATAGCATACATTATACGAAGTTAT (SEQ ID NO: 9), or fragments thereof.

One or more heterologous nucleic acid sequences can be inserted into the nucleic acid sequence comprising the cryptic splice donor site. For example, the nucleic acid sequence comprising the cryptic splice site can be mutated by inserting about 2 to 20 (e.g., about 2, 5, 10, 15, 20, or ranges thereof) heterologous nucleic acid sequences therein. The heterologous nucleic acid sequence can be inserted at any location within the nucleic acid sequence comprising the cryptic splice donor site. In one embodiment, the heterologous nucleic acid sequence can be inserted within an open reading frame (ORF) of the nucleic acid sequence comprising a cryptic splice donor site. For example, the heterologous nucleic acid sequence can be inserted upstream or downstream of a cryptic splice site. In embodiments where the nucleic acid sequence comprising the cryptic splice donor site is mutated by inserting multiple heterologous nucleic acid sequences therein, the heterologous nucleic acid sequences can be inserted upstream and/or downstream of the cryptic splice donor site. For example, about 2-20 (e.g., about 2, 5, 10, 15, 20, or ranges thereof) heterologous nucleic acid sequences can be inserted upstream of the cryptic splice donor site. In addition or alternatively, about 2-20 (e.g., about 2, 5, 10, 15, 20, or ranges thereof) heterologous nucleic acid sequences can be inserted downstream of the cryptic splice donor site. In addition, the heterologous nucleic acid sequence can be inserted into the 5′ or 3′ untranslated regions of the nucleic acid sequence comprising the cryptic splice donor site.

In a preferred embodiment of the invention, the nucleic acid sequence comprising the cryptic splice donor site is mutated by inserting a first heterologous nucleic acid sequence upstream of the cryptic splice donor site, and inserting a second heterologous nucleic acid sequence downstream of the cryptic splice donor site. In another preferred embodiment, the nucleic acid sequence comprising the cryptic splice donor site is mutated by inserting a first heterologous nucleic acid sequence within an open reading frame of the nucleic acid sequence and inserting a second heterologous nucleic acid sequence within a 3′ untranslated region of the nucleic acid sequence.

In a preferred embodiment, the nucleic acid sequence comprises, from 5′ to 3′: (a) a cryptic splice donor site incorporated within an open reading frame of the nucleic acid sequence; (b) a first heterologous nucleic acid sequence incorporated within the open reading frame of the nucleic acid sequence; (c) a second heterologous nucleic acid sequence incorporated within the 3′ untranslated region; and (d) a splice acceptor site incorporated within the 3′ untranslated region. In yet another preferred embodiment, the nucleic acid sequence comprises, from 5′ to 3′: (a) a first heterologous nucleic acid sequence incorporated within an open reading frame of the nucleic acid sequence; (b) a cryptic splice donor site incorporated within the open reading frame of the nucleic acid sequence; (c) a second heterologous nucleic acid sequence incorporated within the 3′ untranslated region; and (d) a splice acceptor site incorporated within the 3′ untranslated region.

In the context of the inventive method, the nucleic acid sequence comprising a cryptic splice donor site is mutated to provide a mutant nucleic acid sequence that has “modified” splice site usage profile, in that the mutant nucleic acid sequence has a splice site usage profile that differs from the splice site usage profile of the nucleic acid sequence prior to mutation. The term “splice site usage profile,” as used herein, refers to the frequency with which particular splice donor and splice acceptor sites within a nucleic acid sequence are utilized to produce specific mRNA transcripts. The splice site usage profile of the mutant nucleic acid sequence “differs” from that of the nucleic acid sequence prior to mutation if the splicing machinery utilizes at least one splice donor site or splice acceptor site that is not utilized in the nucleic acid sequence prior to mutation. Alternatively, the splice site usage profile of the mutant nucleic acid sequence differs from that of the nucleic acid sequence prior to mutation if the splicing machinery utilizes at least one splice donor site or splice acceptor site that is utilized in the nucleic acid sequence prior to mutation, but with greater efficiency in the mutant nucleic acid sequence as compared to the nucleic acid sequence prior to mutation.

The invention also provides an isolated nucleic acid sequence encoding a gene product of interest, wherein the nucleic acid sequence comprises: (a) a cryptic splice donor site (b) a heterologous nucleic acid sequence; and (c) a splice acceptor site; wherein at least two different transcripts are produced when the nucleic acid sequence is introduced into a cell. The descriptions of the cryptic splice donor site, the heterologous nucleic acid sequence, and the splice acceptor site as described herein with respect to the inventive method for preparing a nucleic acid sequence also apply to those same features of the isolated nucleic acid sequence. The nucleic acid sequence is “isolated” in that it is removed from its natural environment.

The nucleic acid sequence encodes a gene product of interest, which can be an RNA molecule (e.g., mRNA or tRNA) or a polypeptide (also referred to herein as a “protein”). Examples of suitable proteins include, for example, surface proteins, intracellular proteins, membrane proteins, and secreted proteins from any unmodified or synthetic source. The gene product of interest preferably is an antibody heavy chain or portion thereof, an antibody light chain or portion thereof, an enzyme, a receptor, a structural protein, a co-factor, a polypeptide, a peptide, an intrabody, a selectable marker, a toxin, a growth factor, or a peptide hormone. The invention also provides a protein generated by expression of the nucleic acid sequence comprising the cryptic splice donor site described herein.

The gene product of interest can be any suitable enzyme, including enzymes associated with microbiological fermentation, metabolic pathway engineering, protein manufacture, bio-remediation, and plant growth and development (see, e.g., Olsen et al., Methods Mol. Biol., 230: 329-349 (2003); Turner, Trends Biotechnol., 21(11): 474-478 (2003); Zhao et al., Curr. Opin. Biotechnol., 13(2): 104-110 (2002); and Mastrobattista et al., Chem. Biol., 12(12): 1291-300 (2005)).

The gene product of interest can be an antigen. An “antigen” is any molecule that induces an immune response in a mammal. An “immune response” can entail, for example, antibody production and/or the activation of immune effector cells (e.g., T-cells). An antigen in the context of the invention can comprise any subunit, fragment, or epitope of any proteinaceous or non-proteinaceous (e.g., carbohydrate or lipid) molecule which provokes an immune response in mammal. By “epitope” is meant a sequence on an antigen that is recognized by an antibody or an antigen receptor. Epitopes also are referred to in the art as “antigenic determinants.”

In a preferred embodiment of the invention, the gene product of interest is an antibody or a portion thereof. For example, the gene product of interest can be an antibody heavy chain or portion thereof or an antibody light chain or portion thereof. The nucleic acid sequence can encode an antibody, or fragment thereof, directed against any suitable antigen. Nucleic acid sequences encoding all naturally occurring germline, affinity matured, synthetic, or semi-synthetic antibodies, as well as fragments thereof, can be used in the present invention. The gene product can be any suitable antibody fragment, such as, e.g., F(ab′)2, Fab′, Fab, Fv, scFv, dsFv, dAb, or a single chain binding polypeptide. The antibody, or fragment thereof, desirably is a mammalian antibody (e.g., a human antibody or a non-human antibody). Preferably, the antibody is a human antibody. A human antibody, a non-human antibody, or a chimeric antibody can be obtained by any means, including in vitro sources (e.g., a hybridoma or a cell line producing an antibody recombinantly) and in vivo sources (e.g., rodents). Methods for generating antibodies are known in the art and are described in, for example, see, e.g., Köhler and Milstein, Eur. J. Immunol., 5: 511-519 (1976); Harlow and Lane (eds.), Antibodies: A Laboratory Manual, CSH Press (1988); and C. A. Janeway et al. (eds.), Immunobiology, 5th Ed., Garland Publishing, New York, N.Y. (2001)). In certain embodiments, a human antibody or a chimeric antibody can be generated using a transgenic animal (e.g., a mouse) wherein one or more endogenous immunoglobulin genes are replaced with one or more human immunoglobulin genes. Examples of transgenic mice wherein endogenous antibody genes are effectively replaced with human antibody genes include, but are not limited to, the HUMAB-MOUSE™, the Kirin TC MOUSE™, and the KM-MOUSE™ (see, e.g., Lonberg N., Nat. Biotechnol., 23(9): 1117-25 (2005); and Lonberg N., Handb. Exp. Pharmacol., 181: 69-97 (2008)).

In some embodiments, such antibody-encoding sequences can be altered through somatic hypermutation (SHM) to create affinity-matured antibody sequences. As used herein, “somatic hypermutation” or “SHM” refers to the mutation of a polynucleotide sequence which can be initiated by, or associated with, the action of activation-induced cytidine deaminase (AID), which includes members of the AID/APOBEC family of RNA/DNA editing cytidine deaminases that are capable of mediating the deamination of cytosine to uracil within a DNA sequence (see, e.g., Conticello et al., Mol. Biol. Evol., 22: 367-377 (2005), and U.S. Pat. No. 6,815,194). SHM can also be initiated by, or associated with, for example, the action of uracil glycosylase and/or error prone polymerases on a polynucleotide sequence of interest. SHM is intended to include mutagenesis that occurs as a consequence of the error prone repair of an initial DNA lesion, including mutagenesis mediated by the mismatch repair machinery and related enzymes. Systems and methods for inducing somatic hypermutation, including nucleic acid and amino acid sequences encoding AID, are described in, e.g., International Patent Application Publication Nos. WO 2008/103475, WO 2008/103474, WO 2003/095636, and U.S. Provisional Patent Application No. 61/166,349.

The gene product of interest also can be a fusion protein (also referred to in the art as a “chimeric protein”). Fusion proteins are generated by transcriptionally linking two or more nucleic acid sequences which code for separate proteins. Translation of the linked genes produces a single polypeptide with functional properties derived from each of the individual proteins. In the context of the invention, the fusion protein can be naturally-occurring (e.g., antibody proteins or the bcr-abl fusion protein), or the fusion protein can be synthetically generated using recombinant DNA techniques known in the art. For example, a nucleic acid sequence encoding a peptide tag can be ligated to a second nucleic acid sequence encoding a gene product of interest to facilitate protein purification and/or identification. Suitable peptide tags include, for example, a glutathione-S-transferase (GST) protein, a FLAG peptide, or a polyhistidine (HIS) tag. Fc fusion proteins are another type of synthetic fusion protein that can be used in the invention. Fc fusion proteins contain a soluble antibody constant fragment (Fc). Soluble Fc fusion proteins can be used as reagents for several in vitro and in vivo applications, including, but not limited to, immunotherapy, flow cytometry, immunohistochemistry, and in vitro activity assays. Fc fusion proteins are described in, for example, Flanagan et al., “Soluble Fc Fusion Proteins for Biomedical Research,” In: M. Albitar, ed., Monoclonal Antibodies: Methods and Protocols (Methods in Molecular Biology), Human Press, Inc., pp. 33-52 (2008). The fusion protein can be used for therapeutic or diagnostic purposes. For example, a therapeutic fusion protein can be generated in which one portion of the fusion protein is capable of directing the fusion protein to a specific cell or tissue, while the other portion of the fusion protein is a biologically active protein or peptide (also referred to in the art as a “payload”), such as an antibody or a cytotoxic protein.

It will be appreciated that the efficiency of splicing depends on a variety of factors, such as, for example, the strength and sequence context of the splice donor and/or acceptor sites, as well as the expression levels of certain splicing factors. Thus, in some embodiments of the invention, splicing efficiency of the nucleic acid sequence comprising the cryptic splice donor site will be less than 100%. For example, at least 10% (e.g., at least 15%, 20%, 25%, 30%, 35%, 40%, 45%, or 50%) of the RNA transcribed from the nucleic acid sequence comprising the cryptic splice donor site is not spliced. In another embodiment, at least 20% (e.g., at least 25%, 30%, 35%, 40%, 45%, 50%, 55%, 60%, 65%, 70%, or 75%), of the RNA transcribed from the nucleic acid sequence comprising the cryptic splice donor site is not spliced. Alternatively, at least 50% (e.g., at least 55%, 60%, 65%, 70%, 75%, 80%, 85%, 90%, or 95%) of the RNA transcribed from the nucleic acid sequence comprising the cryptic splice donor site is not spliced. Preferably, at least 10% (e.g., at least 15%, 20%, 25%, 30%, 35%, 40%, 45%, or 50%) of the RNA transcribed from the nucleic acid sequence comprising the cryptic splice donor site is spliced. More preferably, at least 20% (e.g., at least 25%, 30%, 35%, 40%, 45%, 50%, 55%, 60%, 65%, 70%, or 75%) of the RNA transcribed from the nucleic acid sequence comprising the cryptic splice donor site is spliced. Most preferably, at least 50% (at least 55%, 60%, 65%, 70%, 75%, 80%, 85%, 90%, or 95%, or even 100%) of the RNA transcribed from the nucleic acid sequence comprising the cryptic splice donor site is spliced.

The invention further provides an expression vector comprising the aforementioned nucleic acid sequence comprising a cryptic splice donor site. The term “expression vector,” as used herein, refers to a molecule (typically a nucleic acid molecule) that contains the necessary regulatory sequences to allow transcription and translation of a gene or genes cloned therein. The expression vector can be “episomal.” An “episome” is a vector that is able to replicate in a host cell, and persists as an extrachromosomal segment of DNA within the host cell in the presence of appropriate selective pressure (see, e.g., Conese et al., Gene Therapy, 11: 1735-1742 (2004)). Representative commercially available episomal expression vectors include, but are not limited to, episomal plasmids that utilize Epstein Barr Nuclear Antigen 1 (EBNA1) and the Epstein Barr Virus (EBV) origin of replication (oriP). The vectors pREP4, pCEP4, pREP7, and pcDNA3.1 from Invitrogen (Carlsbad, Calif.), and pBK-CMV from Stratagene (La Jolla, Calif.) represent non-limiting examples of an episomal vector that uses T-antigen and the SV40 origin of replication in lieu of EBNA1 and oriP.

Other suitable vectors include integrating expression vectors, which may randomly integrate into the host cell's DNA, or may include a recombination site to enable the specific recombination between the expression vector and the host cell's chromosomes. Such integrating expression vectors may utilize the endogenous expression control sequences of the host cell's chromosomes to effect expression of the desired protein. Examples of vectors that integrate in a site specific manner include, for example, components of the flp-in system from Invitrogen (Carlsbad, Calif.) (e.g., pcDNA™5/FRT), or the cre-lox system, such as is found in the pExchange-6 Core Vectors from Stratagene (La Jolla, Calif.). Examples of vectors that randomly integrate into host cell chromosomes include, for example, pcDNA3.1 (when introduced in the absence of T-antigen) from Invitrogen (Carlsbad, Calif.), and pCI or pFN10A (ACT) FLEXI™ from Promega (Madison, Wis.).

The expression vector can be a viral vector. Representative commercially available viral expression vectors include, but are not limited to, the adenovirus-based Per.C6 system available from Crucell, Inc. (Leiden, The Netherlands), the lentiviral-based pLP1 from Invitrogen (Carlsbad, Calif.), and the retroviral vectors pFB-ERV plus pCFB-EGSH from Stratagene (La Jolla, Calif.).

The invention also provides an isolated host cell comprising the aforementioned nucleic acid sequence comprising a cryptic splice donor site or the aforementioned expression vector. The nucleic acid sequence can be introduced into any cell that is capable of expressing the nucleic acid sequence, including any suitable prokaryotic or eukaryotic cell. Preferred host cells are those that can be easily and reliably grown, have reasonably fast growth rates, have well characterized expression systems, and can be transformed or transfected easily and efficiently. Examples of suitable prokaryotic cells include, but are not limited to, cells from the genera Bacillus (such as Bacillus subtilis and Bacillus brevis), Escherichia (such as E. coli), Pseudomonas, Streptomyces, Salmonella, and Erwinia. Particularly useful prokaryotic cells include the various strains of Escherichia coli (e.g., K12, HB101 (ATCC No. 33694), DH5α, DH10, MC1061 (ATCC No. 53338), and CC102).

Preferably, the nucleic acid sequence comprising a cryptic splice donor site is introduced into a eukaryotic cell. Suitable eukaryotic cells are known in the art and include, for example, yeast cells, insect cells, and mammalian cells. Examples of suitable yeast cells include those from the genera Hansenula, Kluyveromyces, Pichia, Rhino-sporidium, Saccharomyces, and Schizosaccharomyces. Preferred yeast cells include, for example, Saccharomyces cerivisae and Pichia pastoris. Suitable insect cells are described in, for example, Kitts et al., Biotechniques, 14: 810-817 (1993); Lucklow, Curr. Opin. Biotechnol., 4: 564-572 (1993); and Lucklow et al., J. Virol., 67: 4566-4579 (1993). Preferred insect cells include Sf-9 and HIS (Invitrogen, Carlsbad, Calif.).

Preferably, the isolated host cell is a mammalian cell. A number of suitable mammalian host cells are known in the art, many of which are available from the American Type Culture Collection (ATCC, Manassas, Va.). Examples of suitable mammalian cells include, but are not limited to, Chinese hamster ovary cells (CHO) (ATCC No. CCL61), CHO DHFR-cells (Urlaub et al., Proc. Natl. Acad. Sci. USA, 97: 4216-4220 (1980)), human embryonic kidney (HEK) 293 or 293T cells (ATCC No. CRL1573), and 3T3 cells (ATCC No. CCL92). Other suitable mammalian cell lines are the monkey COS-1 (ATCC No. CRL1650) and COS-7 cell lines (ATCC No. CRL1651), as well as the CV-1 cell line (ATCC No. CCL70). Further exemplary mammalian host cells include primate cell lines and rodent cell lines, including transformed cell lines. Normal diploid cells, cell strains derived from in vitro culture of primary tissue, as well as primary explants also are suitable. Other suitable mammalian cell lines include, but are not limited to, mouse neuroblastoma N2A cells, HeLa, mouse L-929 cells, and BHK or HaK hamster cell lines, all of which are available from the ATCC. Methods for selecting suitable mammalian host cells and methods for transformation, culture, amplification, screening, and purification of such cells are well known in the art (see, e.g., Ausubel et al., eds., Short Protcols in Molecular Biology, 5^{th} J ed., John Wiley & Sons, Inc., Hoboken, N.J. (2002)).

In a preferred embodiment, the mammalian cell is a human cell. For example, the mammalian cell can be a human lymphoid or lymphoid derived cell line, such as a cell line of pre-B lymphocyte origin. Examples of human lymphoid cell lines include, without limitation, RAMOS(CRL-1596), Daudi (CCL-213), EB-3 (CCL-85), DT40 (CRL-2111), 18-81 (Jack et al., Proc. Natl. Acad. Sci. USA, 85: 1581-1585 (1988)), Raji cells (CCL-86), and derivatives thereof.

The nucleic acid sequence comprising a cryptic splice donor site may be introduced into a cell by “transfection,” “transformation,” or “transduction.” “Transfection,” “transformation,” or “transduction,” as used herein, refers to the introduction of one or more exogenous polynucleotides into a host cell by using physical or chemical methods. Many transfection techniques are known in the art and include, for example, calcium phosphate DNA co-precipitation (see, e.g., Murray E. J. (ed.), Methods in Molecular Biology, Vol. 7, Gene Transfer and Expression Protocols, Humana Press (1991)); DEAE-dextran; electroporation; cationic liposome-mediated transfection; tungsten particle-facilitated microparticle bombardment (Johnston, Nature, 346: 776-777 (1990)); and strontium phosphate DNA co-precipitation (Brash et al., Mol. Cell. Biol., 7: 2031-2034 (1987)). Phage or viral vectors can be introduced into host cells, after growth of infectious particles in suitable packaging cells, which are commercially available.

While the inventive method of preparing a nucleic acid sequence with a modified splice site usage profile is performed using a host cell (either in vivo or in vitro), the method also can be performed using a cell-free gene expression system. A “cell-free gene expression system” refers to a composition comprising all of the elements required for transcription and translation of a nucleic acid sequence. Such elements are known in the art and include, for example, RNA polymerase, transcription factors, splicing factors, tRNA molecules, etc. The cell-free gene expression system can be any suitable composition that enables cell-free transcription and translation. For example, the cell-free gene expression system can comprise the transcription and translation machinery of rabbit reticulocytes, wheat germ extract, E. coli, or any other suitable source. Rabbit reticulocytes can translate large mRNA transcripts and carry out post-translational processing, such as glycosylation, phosphorylation, acetylation, and proteolysis. Wheat germ extract is best suited for expression of smaller proteins, and E. coli cell-free extracts are capable of carrying out transcription and translation in the same reaction environment. Commercially available cell-free expression compositions include, for example, rabbit reticulocyte extracts (Promega, Madison, Wis.), pCOLADue™ (Novagen, Madison, Wis.), EXPRESSWAY™ Linear Expression System (Invitrogen Corp., Carlsbad, Calif.), pIEx™ Insect Cell Expression Plasmids (Novagen, Madison, Wis.), and the Rapid Translation System (Roche Diagnostics Corp., Indianapolis, Ind.).

The invention also provides a method of producing an alternate form of an RNA molecule encoded by a nucleic acid sequence. The method comprises (a) preparing a nucleic acid sequence encoding an RNA molecule, wherein the nucleic acid sequence comprises (i) a cryptic splice donor site, (ii) a heterologous nucleic acid sequence, and (iii) a splice acceptor site; and (b) introducing the nucleic acid sequence into a host cell, such that RNA splicing occurs between the cryptic splice donor site and the splice acceptor site to produce an alternate form of the RNA molecule encoded by the nucleic acid sequence. The descriptions of the nucleic acid sequence, the cryptic splice donor site, the heterologous nucleic acid sequence, and the splice acceptor site as described herein with respect to the inventive nucleic acid sequence, or method of preparing same, also apply to those same features of the method of producing an alternate form of RNA. An “alternate form of RNA” is an RNA molecule that would not normally be transcribed from the nucleic acid sequence but for the cryptic splice donor site and heterologous nucleic acid sequence. In other words, an “alternate form of RNA” is an RNA molecule that is produced when the cryptic splice donor site is recognized by the spliceosome after mutation of the nucleic acid sequence (e.g., by insertion of a heterologous nucleic acid sequence) and subsequently activated.

In the context of the inventive method, the alternate form of RNA may be produced to the exclusion of the RNA that is produced when the cryptic splice donor site is inactive (e.g., the RNA produced when the nucleic acid sequence does not comprises a heterologous nucleic acid sequence that activates the cryptic splice donor site (or the “wild-type” RNA molecule)). In other embodiments, both the alternate form of RNA and the wild-type form of RNA are transcribed from the nucleic acid molecule comprising the cryptic splice donor site. In this respect, two or more (e.g., 2, 3, 5, 10, or more) forms of RNA can be transcribed from the nucleic acid sequence comprising the cryptic splice donor site, depending upon the number of cryptic splice donor sites and heterologous nucleic acid sequences located therein. Preferably, the alternate form of mRNA is translated in a cell to produce an alternate form of a protein (such as any of the proteins described herein). Methods for detecting alternatively spliced forms of RNA are known in the art and can be used in the inventive method. Such methods include, for example, computational prediction methods, microarray analysis, and RT-PCR followed by sequencing (see, e.g., Eckhart et al., JBC, 274: 2613-2615 (1999); Ben-Dov et al., JBC, 283: 1229-1233 (2008)).

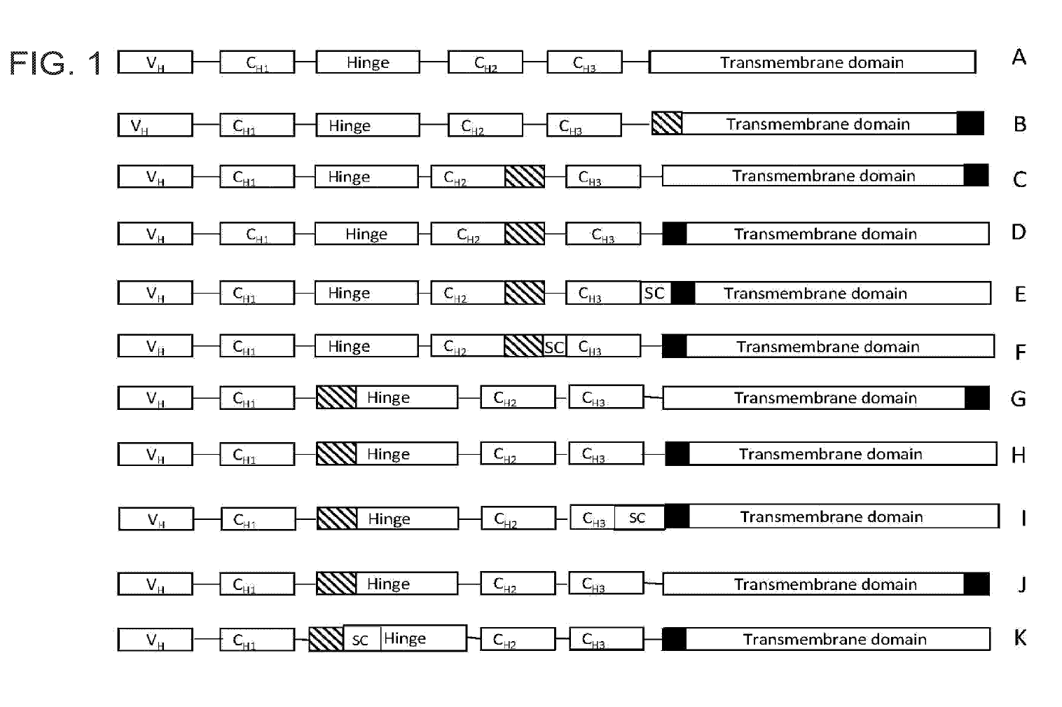

In one embodiment, the inventive method of producing an alternate form of RNA can be used to generate two or more forms of an antibody. For example, the inventive method can be used to generate secreted and membrane-bound forms of the same antibody from a single cell. In addition, the inventive method can be used to generate both full-length antibodies and antibody fragments (such as those described herein) from the same cell. Exemplary strategies for generating secreted or membrane bound antibodies (or fragments thereof) using the inventive method are illustrated in FIG. 1. One of ordinary skill in the art will appreciate that the ability to titrate the amount of secreted and membrane-bound antibodies on the surface of a cell enables the creation of cell lines that exhibit different ratios of secreted and membrane associated antibodies, which may be important when screening for antibody affinity, avidity, and other characteristics. For example, during the early phases of antibody screening it may be desirable to have a higher copy number of antibodies on the cell surface, and a lower copy number of antibodies secreted from the cell in order to maximize low affinity interactions by promoting avidity effects. However, as the screening process progresses, it may be desirable to use cells that secrete more antibody to enable more effective downstream analysis of the secreted antibodies, while reducing the amount of membrane-bound antibodies to reduce avidity effects.

The inventive method of producing an alternate form of RNA also can be used to generate alternate forms of RNA that encode an antigen. For example, the inventive method can be used to produce soluble and membrane-bound forms of an antigen, which optionally can be epitope-tagged (e.g., to confirm the activity of soluble and membrane-bound antigen). Furthermore, the inventive method can be used to generate alternate forms of RNA that encode different forms of the AID protein, which is employed in the SHM methods described herein. For example, the inventive method can be used to generate a C-terminally truncated form of AID with increased activity, a full-length AID protein with reduced activity, or AID proteins with altered cellular localization patterns.

The inventive method of producing an alternate form of RNA also can be used to generate RNA molecules which can be used to interfere with the expression or silence the expression of a particular gene of interest. Such interference may occur through the interruption of transcription, translation, and/or splicing of a particular gene. In one embodiment, the alternate form of RNA binds directly to the DNA and/or RNA encoding a gene of interest, where such binding results in reduced or modified expression of such gene. In another embodiment, the alternate form of RNA can mediate RNA interference (RNAi). RNAi is known in the art as a ubiquitous mechanism of gene regulation in plants and animals in which target mRNAs are degraded in a sequence-specific manner (see, e.g., Sharp, Genes Dev., 15, 485-490 (2001); Hutvagner et al., Curr. Opin. Genet. Dev., 12, 225-232 (2002); Fire et al., Nature, 391, 806-811 (1998); Zamore et al., Cell, 101, 25-33 (2000)). The natural RNA degradation process is initiated by the dsRNA-specific endonuclease Dicer, which promotes cleavage of long dsRNA precursors into double-stranded fragments between 21 and 25 nucleotides long, which are called small interfering RNA (siRNA; also known as short interfering RNA) (see e.g., Zamore, et al., Cell, 101, 25-33 (2000); Elbashir et al., Genes Dev., 15, 188-200 (2001); Hammond et al., Nature, 404, 293-296 (2000); Bernstein et al., Nature, 409, 363-366 (2001)). siRNAs are incorporated into a large protein complex that recognizes and cleaves target mRNAs (Nykanen et al., Cell, 107, 309-321 (2001). The term “siRNA” as used herein, refers to an RNA (or RNA analog) comprising from about 10 to about 50 nucleotides (or nucleotide analogs), which is capable of directing or mediating RNAi. In preferred embodiments, an siRNA molecule comprises about 15 to about 30 nucleotides (or nucleotide analogs) or about 20 to about 25 nucleotides (or nucleotide analogs), e.g., 21-23 nucleotides (or nucleotide analogs). The siRNA can be double or single stranded, but preferably is double-stranded. The use of siRNA as therapeutics for specific disease targets is disclosed in, for example, U.S. Pat. Nos. 5,898,031; 6,107,094; 6,506,559; 7,056,704; 7,078,196; and 7,432,250.

Alternatively, the alternate form of RNA produced by the inventive method can be a short hairpin RNA (shRNA) that mediates RNAi of a gene of interest. The term “shRNA,” as used herein refers to a nucleic acid molecule of about 20 or more base pairs in which a single-stranded RNA partially contains a palindromic base sequence and forms a double-strand structure therein (i.e., a hairpin structure). An shRNA can be an siRNA (or siRNA analog) which is folded into a hairpin structure. shRNAs typically comprise about 45 to about 60 nucleotides, including the approximately 21 nucleotide antisense and sense portions of the hairpin, optional overhangs on the non-loop side of about 2 to about 6 nucleotides long, and the loop portion that, for example, can be about 3 to 10 nucleotides long.

The following examples further illustrate the invention but, of course, should not be construed as in any way limiting its scope.

### Example 1

This example describes a method of preparing a nucleic acid sequence with a modified splice site usage profile, wherein the nucleic acid sequence encodes a portion of a chimeric antibody heavy chain polypeptide.

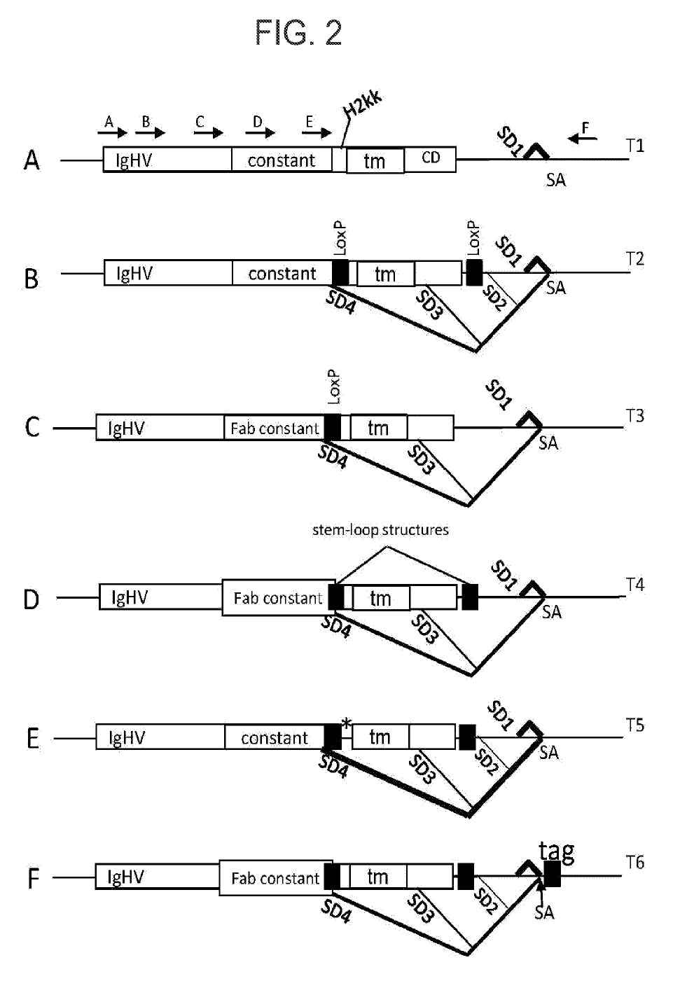

Nucleic acid constructs comprising a nucleic acid sequence encoding the C-terminal region of human IgG1 heavy chain polypeptide, which encodes the constant region of the antibody, were generated using the methods disclosed in U.S. Patent Application Publication No. 2009/0093024 A1. The H2kk peritransmembrane, transmembrane, and cytoplasmic domains were appended to the human IgG1 heavy chain constant region (not including the stop codon) to generate a chimeric immunoglobulin gene. The resulting chimeric protein encodes an IgG1 immunoglobulin molecule that is retained on the cell surface and is able to bind a proteinaceous antigen. The nucleic acid sequence encoding the aforementioned human IgG1 heavy chain (referred to as T1 in FIG. 2) is approximately 2.4 kb in length. The T1 gene contains a known splice donor (SD1) and splice acceptor pair in its 3′ region.

The aforementioned chimeric immunoglobulin gene was modified by insertion of two LoxP domains (as described in U.S. Patent Application Publication No. 2009/0093024 A1) indicated with black boxes on either side of the transmembrane domain (referred to as T2 in FIG. 2). In addition to the known splice donor (SD1) and splice acceptor in the 3′ region of T2, several additional cryptic splice donor sites (SD3 and SD4 in FIG. 2) were unmasked by insertion of the two LoxP domains mentioned above.

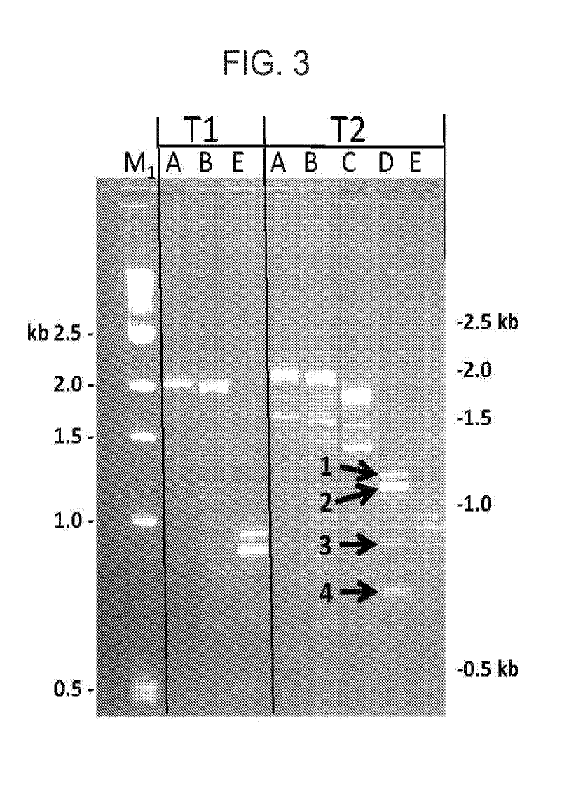

The T1 and T2 constructs were each transfected into HEK293 cells, and RNA transcripts of T1 and T2 were separately converted to DNA using RT-PCR and amplified using primers (A, B, C, D, or E in combination with F as illustrated in FIG. 3). Amplified DNA was subsequently analyzed by gel electrophoresis. Transfection of templates into HEK293 cells was performed as follows. Twenty hours before transfection, HEK293 were plated at 2e5 cells/mL in DMEM/10% fetal bovine serum (DMEM and FBS from Invitrogen, Carlsbad, Calif.). Three μL Fugene 6 (Roche) was added to 100 μL of Optimem (Invitrogen) with vortexing and incubated at room temperature for 5 minutes. Plasmid DNA (1 μg) was added to the Fugene/Optimem mix and allowed to incubate for 25 minutes at room temperature. The DNA-containing mixture was added to 2 mL of HEK293 that were plated the previous day. At 48 hrs, cells were trypsinized and plated into a T75 with media containing DMEM, 10% FBS, and 400 μg/mL G418. One day later, cells were plated with selection media containing DMEM, 10% FBS, 400 μg/mL G418, 2.5 mL gentamycin (Invitrogen), 200 μg/mL hygromycin, and 1 μg puromycin. RNA was harvested from cells following 12 to 14 days of growth using an RNeasy kit from Qiagen (Valencia, Calif.) following the manufacturer's suggested protocol. RT-PCR was performed using Invitrogen's Superscript 3 as per the manufacturer's protocol. Following reverse transcription with random hexamer primers, PCR reaction was accomplished using oligo primers GCCACCATGGAGTTTGGGCTGA (forward, ATG represents the open reading frame's start codon) (SEQ ID NO: 10) and CTATTACTAAACACAGCATG (reverse) (SEQ ID NO: 11) at 1 cycle of 95° C. for 1 hour and 30 minutes; then 25 cycles of 95° C.×45 minutes, 55° C. for 1 hour, 68° C. for variously 90 minutes or 130 minutes (depending on length of expected DNA fragments), and 1 cycle of 68° C. for 5 hours. PCR products were run on a 1% agarose gel; gel bands were purified using Zymoclean (Zymo Research, Orange, Calif.). DNA fragments were TOPO™ cloned using Invitrogen's TOPO™ TA cloning kit as per the kit protocol. Ligation products were transformed into E. coli TOP10™ cells as per the manufacturer's protocol (provided by Invitrogen). Resulting bacterial colonies were prepared using miniprep kits from Qiagen and DNA was sent to Eton Biosciences (San Diego, Calif.) for sequencing.

FIG. 3 illustrates the size(s) of various DNA fragments generated by amplification of the mRNA expressed in HEK293 from the T1 and T2 genes. In lane T1A, strong bands of 2076 base pairs (bp) and 2010 bp were visible. These bands, amplified by oligos A and F, represent cDNA derived from unspliced mRNA and mRNA in which the 66 bp intron bounded by SD1 and SA was spliced out, respectively. One or a few additional very weak bands were also present but barely visible on the gel. Products in lane T2B, PCR amplified using oligos B and F, were 39 nucleotides (nt) shorter than in lane T2A (oligo B lies 39 nt 3′ to oligo A on the amplicon). Similarly, DNA fragments in lane TiE were expected to be 1123 nucleotides shorter than their counterparts in lane T1A, and appeared to migrate at the expected sizes of 953 and 887 bp.

In the case of T1, amplification using primer E clearly indicated the presence of two different DNA fragment sizes that were generated by RNA splicing at the 3′ splice donor (SD1) and splice acceptor in the T1 gene. In the case of T2, various different DNA fragments were generated due to unmasking of various different splice donor sites (including SD4, SD3, and SD2) in the T2 gene sequence. These DNA fragments are indicated with arrows in FIG. 3.

The results of this example confirm that a method of preparing a nucleic acid sequence encoding a IgG1 heavy chain protein comprising inserting two heterologous nucleic acid sequences therein, which results in the modification of the splice site usage profile of the nucleic acid sequence, can be carried out in accordance with the invention.

### Example 2

This example describes a method of preparing a nucleic acid sequence with a modified splice site usage profile, wherein the nucleic acid sequence encodes a portion of a chimeric antibody heavy chain polypeptide.

The T1 gene was generated as outlined in Example 1. A T3 gene was generated as illustrated in FIG. 2 by insertion of a single LoxP sequence upstream of the transmembrane domain, but in the context of a Fab constant domain. The IgG and Fab constructs differ from each other in the presence (IgG) or absence (Fab) of certain amino acids of the IgG1 constant domain. As a result of the single heterologous LoxP site in T3, multiple splice donor sites were unmasked (SD4, SD3, and SD2) and resulted in alternative splicing of the T3 gene into the various different DNA fragments (generated from transfection in HEK293 and primer based amplification in the same manner as that outlined in Example 1).

The results of this example confirm that a method of preparing a nucleic acid sequence encoding a IgG1 heavy chain protein comprising inserting one heterologous nucleic acid sequence therein, which results in the modification of the splice site usage profile of the nucleic acid sequence, can be carried out in accordance with the invention.

### Example 3

This example describes a method of preparing a nucleic acid sequence with a modified splice site usage profile by generating a variety of heterologous stem-loop structures within the nucleic acid sequence.

The T2 gene was generated as outlined in Example 2. A T4 gene was generated by replacing each of the LoxP sites in T2 (SEQ ID NO: 12) with either of two other stem-loop structures (SEQ ID NO: 13 or SEQ ID NO: 14, see FIG. 2) as shown as black boxes in FIG. 2. SEQ ID NO: 12 consists of a 13 nucleic acid stem followed by an 8 nucleic acid loop followed by another 13 nucleic acid stem that is complementary to the first stem. SEQ ID NO: 13 consists of a 10 nucleic acid stem followed by a 9 nucleic acid loop followed by another 10 nucleic acid stem that is complementary to the first stem. SEQ ID NO: 14 consists of a 13 nucleic acid stem followed by an 8 nucleic acid loop followed by a 13 nucleic acid stem that is not complementary to the first stem and hence contains a fewer number of hydrogen bonds between the two stems.

Expression of the T4 gene containing either combination of SEQ ID NO: 13 or SEQ ID NO: 14 as alternatives to a heterologus LoxP site resulted in alternative splicing of the gene to create multiple DNA fragments in accordance with the amplification described in the above examples.

The results of this example confirm that a method of preparing a nucleic acid sequence comprising inserting a heterologous nucleic acid sequence therein, which results in the modification of the splice site usage profile of the nucleic acid sequence, can be carried out in accordance with the invention.

### Example 4

This example describes a method of producing an alternate form of an RNA molecule encoding an antibody heavy chain protein.

The T1 and T2 genes were generated as described in the examples above. A T5 gene was generated by modifying T2 such that a 36-nucleotide sequence between the first LoxP site and the transmembrane domain is deleted.

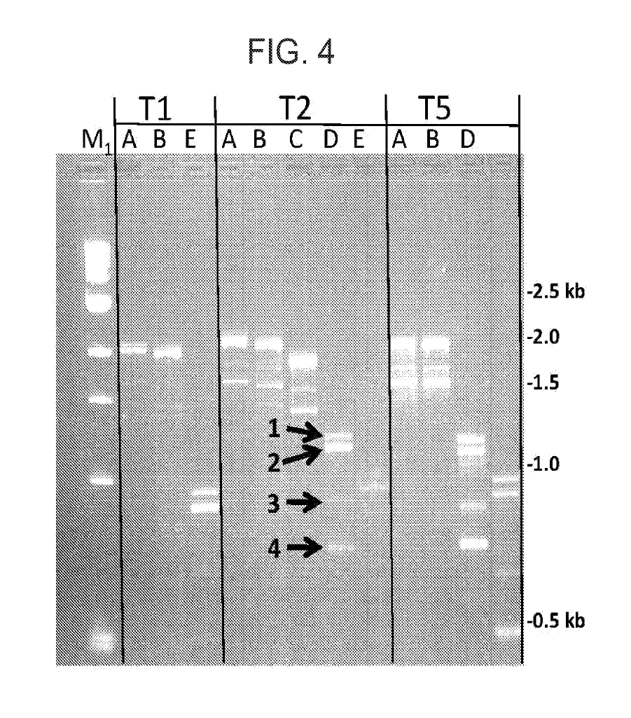

When T2 and T5 were expressed in HEK293 cells, alternative splicing of the genes between SD4 and the 3′ splice acceptor (SA) resulted in removal of the transmembrane domain from the mRNA such that the translated antibody heavy chain paired with its corresponding light chain and was secreted by the cell. This secreted heavy chain corresponds with splice form #4 (approximately 0.75 kb) on the agarose gel in FIG. 4. The deletion of 36 nucleic acids from T2 to generate T5 lead to increased expression of secreted antibody when transfected into HEK293 cells.

The results of this example confirm that a method of producing an alternate form of an RNA molecule encoding an antibody heavy chain protein which is secreted from a cell can be carried out in accordance with the invention.

### Example 5

This example describes a method of producing an alternate form of an RNA molecule encoding a His- and FLAG-tagged antibody heavy chain fusion protein.

The T6 gene was created by modifying the T2 gene described above to insert a His tag (HHHHHHHHH (SEQ ID NO: 15)) or FLAG (DYKDDDDKG (SEQ ID NO: 16)) fusion domain at the 3′ end of the gene immediately following the splice acceptor (SA) site.

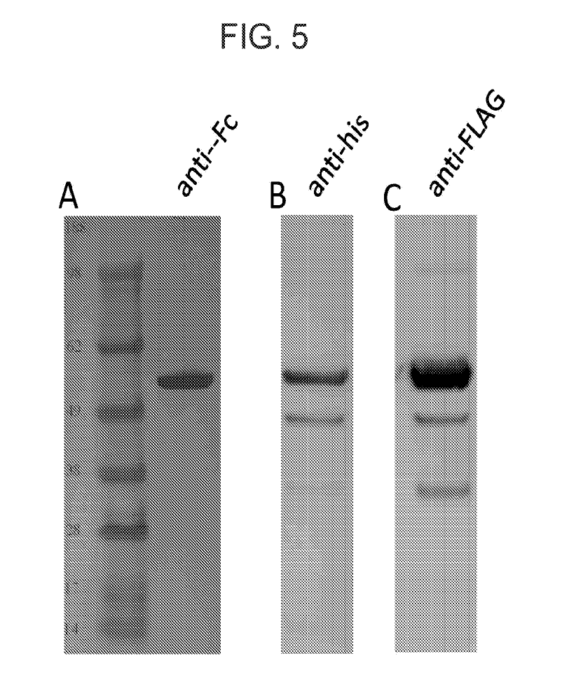

When expressed in HEK293 cells (as described above), the T6 gene underwent splicing at SD2, SD3, and SD4 (where these splice donor sites are unmasked by insertion of the LoxP sequences indicated in black in FIG. 2) such that the spliced mRNA fuses the His or FLAG coding regions immediately following (i.e. 3′ to) the excised SD2-, SD3-, or SD4- to the SA intron. Such splicing resulted in expression of protein that can be detected by Western blotting by staining with anti-Fc antibody (in the case of control heavy chain), anti-His antibody, or anti-FLAG antibody (in the case of heavy chain sequences where a 3′ tag has been inserted). Western blotting under reducing conditions was performed as follows: 10 μL LDS sample buffer (4×) (Invitrogen, NP0007) and 4 μl reducing agent (10×) (Invitrogen, NP0004) were mixed with 26 μA of each sample. The mixture was heated at 70° C. for 10 minutes, and samples were then loaded onto a 4-12% Bis-Tris mini-gel (15 mm×10 well, Invitrogen, NPO335BOX) adjacent to a lane loaded with See Blue Plus2 prestained standard (1×) (Invitrogen, LC5925). The gel was run for approximately 40 minutes, and samples were then transferred to a mini nitrocellulose membrane (Invitrogen, IB3010-02) for 7 minutes by using the iBLOT™ gel transfer system (Invitrogen, IB1001). Following the transfer, the membrane was washed with 20 mL distilled water twice for 5 minutes on a rotary shaker. The membrane was then incubated with 10 mL block buffer for 30 minutes, and washed again with 20 mL distilled water for 5 minutes. The membrane was subsequently incubated with 10 mL block buffer plus 1:5000 dilution of antibody (anti-his or anti-flag as appropriate for each set of lanes) for 1 hour. The membrane was washed with 20 mL antibody wash solution 3 times for 5 minutes, then with 20 mL distilled water twice for 2 minutes. Finally, 10 mL of HRP chromogenic substrate (TMB) (Invitrogen, WP20004) was added to develop the membrane, which is photographed using a Fluor Chem camera (Alpha Innotech, San Leandro, Calif.). The results of the Western blot are illustrated in FIG. 5.

The results of this example confirm that a method of producing an alternate form of an RNA molecule encoded by a nucleic acid sequence encoding a gene product of interest can be carried out in accordance with the invention.

### Example 6

This example describes a method of preparing an alternate form of an RNA molecule encoding an antibody heavy chain protein.

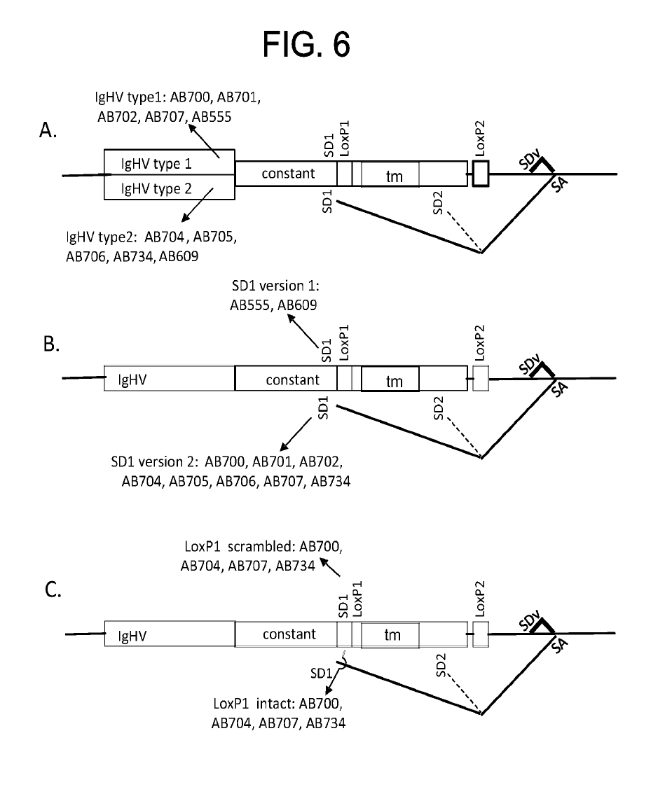

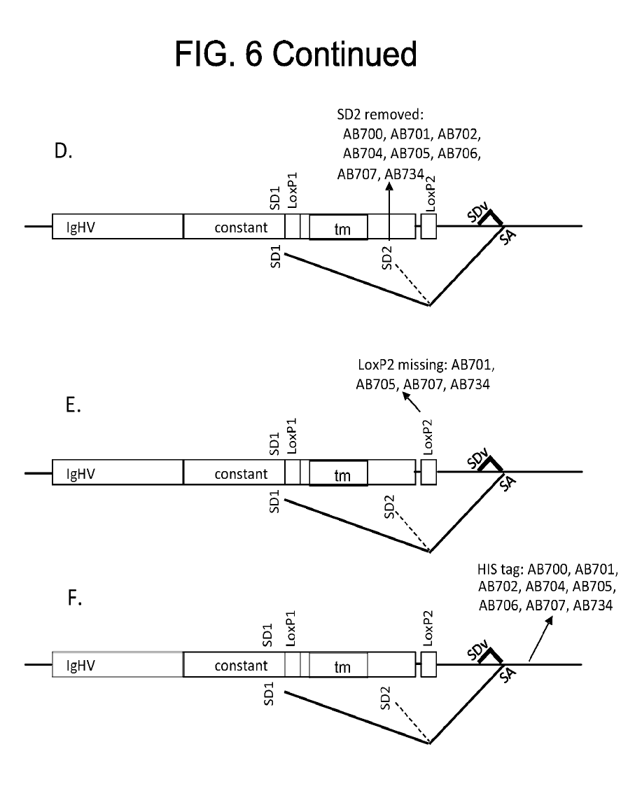

Nucleic acid constructs comprising a nucleic acid sequence encoding the variable (IgHV) and Fab constant regions of a human IgG1 heavy chain polypeptide were generated using the methods disclosed in U.S. Patent Application Publication Nos. 2009/0093024 A1 and 2009/0075378 A1. The H2kk transmembrane, peritransmembrane, and cytoplasmic domains were appended to the human IgG1 heavy chain constant region (not including the stop codon). The constructs were modified by insertion of either one or two LoxP domains (as described in U.S. Patent Application Publication No. 2009/0093024 A1) on either side of the H2kk transmembrane domain, and/or the insertion of a His tag at the C-terminus, as set forth below in Table 1 (see also FIGS. 6A-6F). In addition to the known splice donor (SD1) and splice acceptor (SA) in the 3′ regions of the nucleic acid sequences, an additional cryptic splice donor (SD2) site was unmasked by insertion of the one or two LoxP domains mentioned above (see FIGS. 6A-6F). The resulting nucleic acid constructs encode an IgG1 immunoglobulin molecule that is retained on the cell surface, or the constructs can undergo alternative splicing to generate an immunoglobulin molecule that is secreted from the cell.

TABLE 1

| | Known Splice | # of | SD2 | His |
|---|---|---|---|---|
| | Donor Sequence | Loxp | present | Tag |
| Construct | (SD1) | Sites | (Y/N) | (Y/N) |
| AB609 | CTTGTGACA | 2 | Y | N |
| | (SEQ ID NO: 17) | | | |
| AB555 | SEQ ID NO: 17 | 2 | Y | N |
| AB706 | CAGGTAAAT | 2 | N | Y |
| | (SEQ ID NO: 18) | | | |
| AB702 | SEQ ID NO: 18 | 2 | N | Y |
| AB704 | SEQ ID NO: 18 | 2 | N | Y |
| AB700 | SEQ ID NO: 18 | 2 | N | Y |
| AB705 | SEQ ID NO: 18 | 1 | N | Y |
| AB701 | SEQ ID NO: 18 | 1 | N | Y |
| AB734 | SEQ ID NO: 18 | 1 | N | Y |
| AB707 | SEQ ID NO: 18 | 1 | N | Y |

As a result of the insertion of heterologous LoxP sites in these constructs, the mRNA transcribed from the DNA constructs (generated from transfection in HEK293 cells and primer based amplification in the same manner as that outlined in Example 1) can be alternatively spliced. The unspliced mRNAs encode a cell surface-associated antibody that contains a transmembrane domain. The alternatively spliced mRNAs encode a secreted version of the same antibody in which the transmembrane domain and some surrounding sequences have been removed.

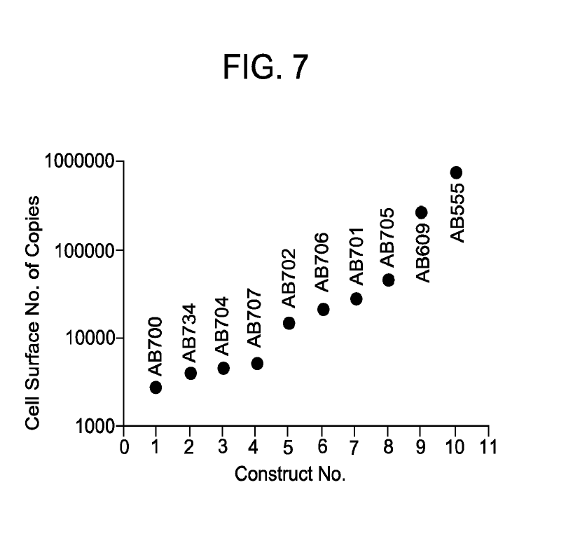

The approximate average Fab retained on the surface per cell was determined for the constructs described above (see FIG. 7). Average surface Fab density was calculated using Quantum™ Alexa Fluor® 647 MESF microspheres (Bang Laboratories, Inc., Fishers, In) as per the manufacturer's suggested protocol following cell separation on a Cytopeia INFLUX cell sorter (BD Biosciences, San Jose, Calif.). The results of this assay are set forth in Table 2. All tested constructs were able to undergo alternative splicing to generate a secreted form of the same antibody.

TABLE 2

| | Molecules of Soluble | |
|---|---|---|
| | Fluorescence Units | |
| Construct | (MESF) | Sites per Cell |
| HEK293 c18 (control) | 21,592 | control +/− 1904 |
| AB704/337 | 24,506 | 4,680 |
| AB705/337 | 66,794 | 47,102 |
| AB706/337 | 41,078 | 21,508 |
| AB734/337 | 23,760 | 4,214 |
| AB609/337 | 285,858 | 266,394 |
| AB700/326 | 23,383 | 2,920 |
| AB701/326 | 48,729 | 28,488 |
| AB702/326 | 35,734 | 15,152 |
| AB707/326 | 24,943 | 5,271 |
| AB555/326 | 785,461 | 765,564 |

The results of this example confirm that a method of producing an alternate form of an RNA molecule encoding an antibody can be carried out in accordance with the invention.

### Example 7

This example demonstrates the functional activity of a secreted antibody molecule encoded by an alternate form of RNA produced in accordance with the invention.

Two nucleic acid sequences (SEQ ID NO: 19 and SEQ ID NO: 22) encoding secreted versions of an antibody which binds interleukin-17 (IL-17) were generated using the methods described in Examples 5 and 6. The functional activity of these antibodies (“engineered antibodies”) was compared to the activity of three control anti-IL-17 antibodies with known functional activity in this assay.

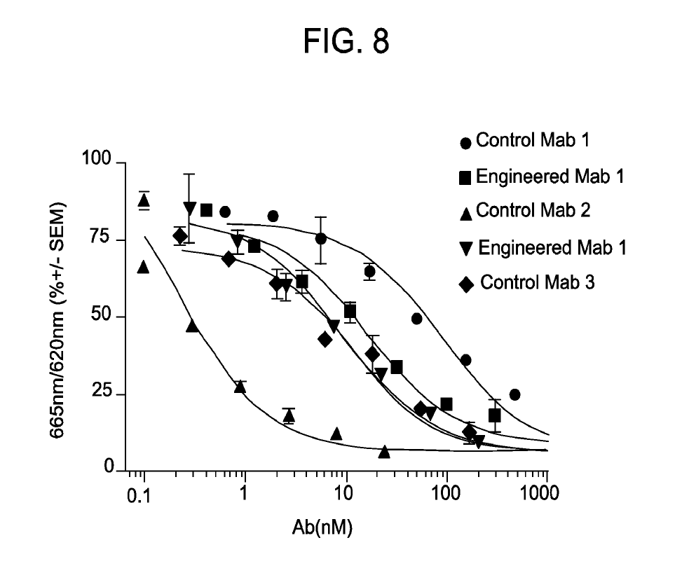

The binding affinity rank order of the IL-17 antibodies was determined by a homogenous time-resolved fluorescence (HTRF) assay. In the assay, the antigen (IL-17-tagged with wasabi fluorescent protein (wfp)) was labeled with N-hydroxysuccinimide-activated Cryptate (Eu3+-TBP-NHS Cryptate) using a HTRF® Cryptate Labeling Kit following the manufacturer's protocol (Cisbio Bioassays Bedford, Mass.). To perform the assay, a reference antibody was biotinylated, mixed with SA-XL665 (Cisbio Bioassays Bedford, Mass.), and then mixed with an unlabeled test antibody at varying concentrations. The antibodies were then incubated with the labeled antigen overnight at room temperature. After incubation, the reaction was read in a ProxiPlate-384 Plus (Perkin Elmer, Waltham, Mass.) using an Envision plate reader. The binding of the labeled antigen and the reference antibody was determined as the ratio of 665 nm to 620 nm. The ratios were plotted against the concentrations of the test antibodies, and the IC₅₀s were determined by inhibitory curve fitting using Graphpad Prism. The IC₅₀ values are shown in Table 3. The results of this assay are shown in FIG. 8.

TABLE 3

| | DNA Sequence | Amino Acid Sequence | |
|---|---|---|---|
| Antibody | (SEQ ID NO) | (SEQ ID NO) | IC₅₀ |
| Control Antibody 1 | N/A | N/A | 88 |
| Engineered Antibody 1 | 19 | 20 (unspliced); | 17 |
| | | 21 (spliced | |
| Control Antibody 2 | N/A | N/A | 0.22 |
| Engineered Antibody 2 | 22 | 23 (unspliced); | 8.9 |
| | | 24 (spliced) | |
| Control Antibody 3 | N/A | N/A | 12 |

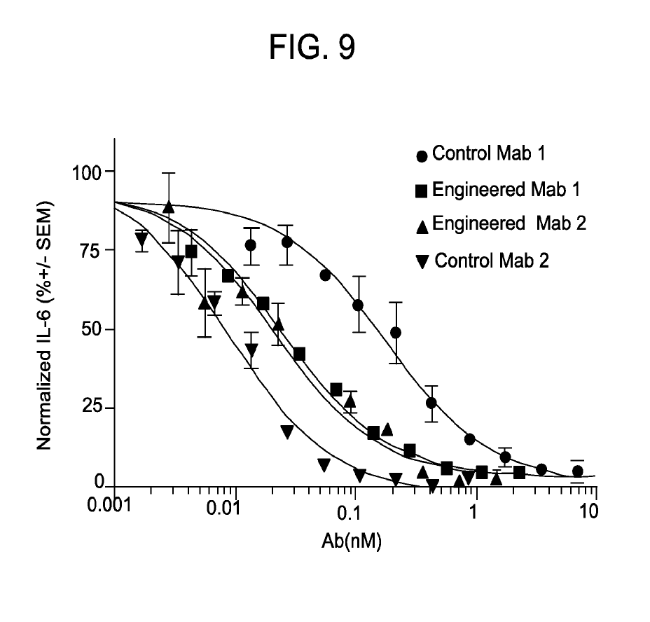

To determine and compare the biological activities of the anti-IL-17 antibodies, IL-17- stimulated IL-6 release from NIH3T3 cells was quantified by ELISA. Specifically, in a 96-well assay plate, 10,000 NIH3T3 cells were plated per well with 0.5 ng/mL human recombinant TNF-α (R&D Systems, Minneapolis, Minn.), purified Myc-tagged human IL-17 (SEQ ID NO: 33), and IL-17 antibodies at varying concentrations in 100′11 DMEM/10% fetal calf serum. The cells were cultured overnight, and 10′11 supernatant from each well was used for ELISA (eBioscience, San Diego, Calif.) to quantify the concentration of interleukin-6 (IL-6). The determined IL-6 levels were plotted against the concentrations of the test antibodies (see FIG. 9), and the IC₅₀s were calculated by inhibitory curve fitting using Graphpad Prism (see Table 4).

TABLE 4

| | Control | Engineered | Control | Engineered |
|---|---|---|---|---|
| | Antibody 1 | Antibody 1 | Antibody 2 | Antibody 2 |
| IC₅₀ | 0.18 | 0.024 | 0.009 | 0.021 |

The results of this example confirm that secreted antibodies expressed by alternatively spliced immunoglobulin gene sequences produced in accordance with the invention are functional.

### Example 8

This example describes a method of generating different secreted forms of the same antibody from an alternatively spliced immunoglobulin gene sequence produced in accordance with the invention.

A nucleic acid construct (SEQ ID NO: 25) is generated, which is tagged alternatively with wasabi fluorescent protein (WFP) or red fluorescent protein (RFP), depending on the splice product that is produced. The nucleic acid construct contains the following elements, from 5′ to 3′: (1) a heavy chain variable region, (2) a IgG1 gamma 1 constant domain, (3) a cryptic splice donor sequence with canonical GT donor site, (4) a first (5′-proximal) LoxP site, (5) a short, flexible gly-ser linker, (6) a WFP sequence, (7) a TGA stop codon for the unspliced (WFP-containing) version of the antibody, (8) the SV40 “little t” intron, (9) a second flexible gly-ser linker, and (10) RFP coding sequence.

Without splicing, the nucleic acid construct will produce a secreted polypeptide containing the WFP tag and lacking the RFP tag (SEQ ID NO: 26). Alternative splicing of the nucleic acid construct utilizing the cryptic GT splice donor site that is unmasked by the Loxp site will result in excision of the WFP sequence and stop codon (SEQ ID NO: 27), and will produce a secreted polypeptide containing the RFP tag and lacking the WFP tag (SEQ ID NO: 28).

The results of this example demonstrate a method of generating different secreted forms of the same antibody from an alternatively spliced immunoglobulin gene sequence produced in accordance with the invention.

### Example 9

This example describes a method of generating fusion proteins using an alternatively spliced DNA sequence produced in accordance with the invention.

A nucleic acid sequence (SEQ ID NO: 29) encoding a fusion protein containing a portion of the HERCEPTIN® IgG antibody and saporin toxin is produced using the methods disclosed herein. The nucleic acid sequence contains the following elements, from 5′ to 3′:

(1) an osteonectin signal peptide, (2) an immunoglobulin heavy chain region (IgHV) from HERCEPTIN®, (3) a IgG1 gamma 1 constant domain, (4) a cryptic splice donor sequence with canonical GT donor site, (5) a first (5′-proximal) LoxP site, (6) the H2kk transmembrane domain, (7) a H2kk peritransmembrane and cytoplasmic domains (positioned 5′ and 3′ to the transmembrane domain, respectively), (8) a TGA stop codon for unspliced version, (9) the SV40 “little t” intron, (10) a flexible gly-ser linker, and (11) a saporin toxin moiety. The saporin toxin sequence (derived from Saponaria officinalis) is obtained from GenBank (nucleotide sequence accession number X59255; amino acid sequence accession number CAA41948). The native signal peptide of saporin toxin is removed.

Without splicing, the nucleic acid sequence will produce a cell membrane-associated fusion protein (SEQ ID NO: 30). Alternative splicing of the nucleic acid construct utilizing the cryptic GT splice donor site unmasked by the Loxp site will result in excision of the transmembrane domain and stop codon (SEQ ID NO: 31), and will produce a secreted fusion protein (SEQ ID NO: 32).

A nucleic acid sequence encoding the Pseudomonas exotoxin A (PE38; GenBank Accession Number 1IKQ_A or AAB59097) or luciferase can be used in place of the saporin toxin nucleic acid sequence in the fusion protein described above.

The results of this example demonstrate a method of generating fusion proteins using an alternatively spliced DNA sequence produced in accordance with the invention.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of preparing a nucleic acid sequence with a modified splice site usage profile, which method comprises:
(a) providing a nucleic acid sequence encoding a gene product of interest, wherein the nucleic acid sequence comprises a cryptic splice donor site and a splice acceptor site; and
(b) mutating the nucleic acid sequence to provide a mutant nucleic acid sequence that has a splice site usage profile that differs from the splice site usage profile of the nucleic acid sequence prior to mutation.

2. The method of claim 1, wherein the cryptic splice donor site is located within an open reading frame of the nucleic acid sequence.

3. The method of claim 1, wherein the cryptic splice donor site is located within a 5′ untranslated region of the nucleic acid sequence.

4. The method of claim 1, wherein the splice site usage profile of the mutant nucleic acid sequence is increased or decreased as compared to the splice site usage profile of the nucleic acid sequence prior to mutation.

5. The method of claim 1, wherein the cryptic splice donor site comprises a GT sequence.

6. The method of claim 1, wherein the cryptic splice donor site is at least 50% identical to CAGGTRAGT, wherein R is A or G.

7. The method of claim 6, wherein the cryptic splice donor site is at least 60% identical to CAGGTRAGT, wherein R is A or G.

8. The method of claim 1, wherein mutating the nucleic acid sequence comprises introducing a point mutation in or adjacent to the cryptic splice donor site.

9. The method of claim 1, wherein mutating the nucleic acid sequence comprises inserting a heterologous nucleic acid sequence upstream of the cryptic splice donor site.

10. The method of claim 9, wherein mutating the nucleic acid sequence comprises inserting two or more heterologous nucleic acid sequences upstream of the cryptic splice donor site.

11. The method of claim 1, wherein mutating the nucleic acid sequence comprises inserting a heterologous nucleic acid sequence downstream of the cryptic splice donor site.

12. The method of claim 11, wherein mutating the nucleic acid sequence comprises inserting two or more heterologous nucleic acid sequences downstream of the cryptic splice donor site.

13. The method of claim 1, wherein mutating the nucleic acid sequence comprises:
(i) inserting a heterologous nucleic acid sequence upstream of the cryptic splice donor site; and
(ii) inserting a heterologous nucleic acid sequence downstream of the cryptic splice donor site.

14. The method of claim 1, wherein the nucleic acid sequence comprises two or more cryptic splice donor sites.

15. The method of claim 1, wherein a splice acceptor site is incorporated within a 3′ untranslated region of the nucleic acid sequence.

16. The method of claim 1, wherein a splice acceptor site is incorporated within an open reading frame of the nucleic acid sequence.

17. The method of claim 1, wherein mutating the nucleic acid sequence comprises inserting a first heterologous nucleic acid sequence within an open reading frame of the nucleic acid sequence and inserting a second heterologous nucleic acid sequence within a 3′ untranslated region of the nucleic acid sequence.

18. The method of claim 17, wherein the nucleic acid sequence comprises, from 5′ to 3′:
(a) the cryptic splice donor site incorporated within an open reading frame of the nucleic acid sequence;
(b) the first heterologous nucleic acid sequence incorporated within the open reading frame of the nucleic acid sequence;
(c) the second heterologous nucleic acid sequence incorporated within the 3′ untranslated region; and
(d) the splice acceptor site incorporated within the 3′ untranslated region.

19. The method of claim 17, wherein the nucleic acid sequence comprises, from 5′ to 3′:
(a) the first heterologous nucleic acid sequence incorporated within an open reading frame of the nucleic acid sequence;
(b) the cryptic splice donor site incorporated within the open reading frame of the nucleic acid sequence;
(c) the second heterologous nucleic acid sequence incorporated within the 3′ untranslated region; and
(d) the splice acceptor site incorporated within the 3′ untranslated region.

20. The method of claim 9, wherein the heterologous nucleic acid sequence forms a stem-loop structure.

21. The method of claim 9, wherein the heterologous nucleic acid sequence encodes a LoxP site.

22. An isolated nucleic acid sequence encoding a gene product of interest, wherein the nucleic acid sequence comprises:
(a) a cryptic splice donor site;
(b) a heterologous nucleic acid sequence; and
(c) a splice acceptor site;
wherein at least two different RNA transcripts are produced when the nucleic acid sequence is introduced into a cell.

23. The isolated nucleic acid sequence of claim 22, wherein the cryptic splice donor site is located within an open reading frame of the nucleic acid sequence.

24. The isolated nucleic acid sequence of claim 22, wherein the cryptic splice donor site is located within a 5′ untranslated region of the nucleic acid sequence.

25. The isolated nucleic acid sequence of claim 22, wherein the cryptic splice donor site comprises a GT sequence.

26. The isolated nucleic acid sequence of claim 22, wherein the cryptic splice donor site is at least 50% identical to CAGGTRAGT, wherein R is A or G.

27. The isolated nucleic acid sequence of claim 26, wherein the cryptic splice donor site is at least 60% identical to CAGGTRAGT, wherein R is A or G.

28. The isolated nucleic acid sequence of claim 22, which comprises a point mutation in or adjacent to the cryptic splice donor site.

29. The isolated nucleic acid sequence of claim 22, wherein the heterologous nucleic acid sequence is located upstream of the cryptic splice donor site.

30. The isolated nucleic acid sequence of claim 29, wherein the nucleic acid sequence comprises two or more heterologous nucleic acid sequences located upstream of the cryptic splice donor site.

31. The isolated nucleic acid sequence of claim 22, wherein the heterologous nucleic acid sequence is located downstream of the cryptic splice donor site.

32. The isolated nucleic acid sequence of claim 31, wherein the nucleic acid sequence comprises two or more heterologous nucleic acid sequences located downstream of the cryptic splice donor site.

33. The isolated nucleic acid sequence of claim 22, wherein the nucleic acid sequence comprises a heterologous nucleic acid sequence located upstream of the cryptic splice donor site and a heterologous nucleic acid sequence located downstream of the cryptic splice donor site.

34. The isolated nucleic acid sequence of claim 22, wherein the nucleic acid sequence comprises two or more cryptic splice donor sites.

35. The isolated nucleic acid sequence of claim 22, wherein the splice acceptor site is located within a 3′ untranslated region of the nucleic acid sequence.

36. The isolated nucleic acid sequence of claim 22, wherein the splice acceptor site is located within an open reading frame of the nucleic acid sequence.

37. The isolated nucleic acid sequence of claim 22, wherein the nucleic acid sequence comprises a first heterologous nucleic acid sequence located within an open reading frame of the nucleic acid sequence and a second heterologous nucleic acid sequence located within a 3′ untranslated region of the nucleic acid sequence.

38. The isolated nucleic acid sequence of claim 37, wherein the nucleic acid sequence comprises, from 5′ to 3′:
(a) the cryptic splice donor site located within an open reading frame of the nucleic acid sequence;
(b) the first heterologous nucleic acid sequence located within the open reading frame of the nucleic acid sequence;
(c) the second heterologous nucleic acid sequence located within the 3′ untranslated region; and
(d) the splice acceptor site located within the 3′ untranslated region.

39. The isolated nucleic acid sequence of claim 37, wherein the nucleic acid sequence comprises, from 5′ to 3′:
(a) the first heterologous nucleic acid sequence located within an open reading frame of the nucleic acid sequence;
(b) the cryptic splice donor site located within the open reading frame of the nucleic acid sequence;
(c) the second heterologous nucleic acid sequence located within the 3′ untranslated region; and
(d) the splice acceptor site incorporated within the 3′ untranslated region.

40. The isolated nucleic acid sequence of claim 22, wherein the heterologous nucleic acid sequence forms a stem-loop structure.

41. The isolated nucleic acid sequence of claim 22, wherein the heterologous nucleic acid sequence encodes a LoxP site.

42. A method of producing an alternate form of an RNA molecule encoded by a nucleic acid sequence encoding a gene product of interest, which method comprises:
(a) preparing a nucleic acid sequence encoding an RNA molecule, wherein the nucleic acid sequence comprises (i) a cryptic splice donor site, (ii) a heterologous nucleic acid sequence, and (iii) a splice acceptor site; and
(b) introducing the nucleic acid sequence into a host cell, such that RNA splicing occurs between the cryptic splice donor site and the splice acceptor site to produce an alternate form of the RNA molecule encoded by the nucleic acid sequence.

43. The method of claim 42, wherein the cryptic splice donor site is located within an open reading frame of the nucleic acid sequence.

44. The method of claim 42, wherein the cryptic splice donor site is located within a 5′ untranslated region of the nucleic acid sequence.

45. The method of claim 42, wherein the cryptic splice donor site comprises a GT sequence.

46. The method of claim 42,
wherein the cryptic splice donor site is at least 50% identical to CAGGTRAGT, wherein R is A or G.

47. The method of claim 46, wherein the cryptic splice donor site is at least 60% identical to CAGGTRAGT, wherein R is A or G.

48. The method of claim 42, wherein the heterologous nucleic acid sequence is incorporated upstream of the cryptic splice donor site.:

49. The method of claim 48, wherein the nucleic acid sequence comprises two or more heterologous nucleic acid sequences incorporated upstream of the cryptic splice donor site.

50. The method of claim 42, wherein the heterologous nucleic acid sequence is incorporated downstream of the cryptic splice donor site.

51. The method of claim 50, wherein the nucleic acid sequence comprises two or more heterologous nucleic acid sequences incorporated downstream of the cryptic splice donor site.

52. The method of claim 42, wherein the nucleic acid sequence comprises a heterologous nucleic acid sequence incorporated upstream of the cryptic splice donor site and a heterologous nucleic acid sequence incorporated downstream of the cryptic splice donor site.

53. The method of claim 42, wherein the nucleic acid sequence comprises two or more cryptic splice donor sites.

54. The method of claim 42, wherein the splice acceptor site is incorporated within a 3′ untranslated region of the nucleic acid sequence.

55. The method of claim 42, wherein the splice acceptor site is incorporated within an open reading frame of the nucleic acid sequence.

56. The method of claim 42, wherein the nucleic acid sequence comprises a first heterologous nucleic acid sequence incorporated within an open reading frame of the nucleic acid sequence and a second heterologous nucleic acid sequence incorporated within a 3′ untranslated region of the nucleic acid sequence.

57. The method of claim 56, wherein the nucleic acid sequence comprises, from 5′ to 3′:
(i) the cryptic splice donor site incorporated within an open reading frame of the nucleic acid sequence;
(ii) the first heterologous nucleic acid sequence incorporated within the open reading frame of the nucleic acid sequence;
(iii) the second heterologous nucleic acid sequence incorporated within the 3′ untranslated region; and
(iv) the splice acceptor site incorporated within the 3′ untranslated region.

58. The method of claim 56, wherein the nucleic acid sequence comprises, from 5′ to 3′:
the first heterologous nucleic acid sequence incorporated within an open reading frame of the nucleic acid sequence;
(ii) the cryptic splice donor site incorporated within the open reading frame of the nucleic acid sequence;
(iii) the second heterologous nucleic acid sequence incorporated within the 3′ untranslated region; and
(iv) the splice acceptor site incorporated within the 3′ untranslated region.

59. The method of claim 42, wherein the heterologous nucleic acid sequence forms a stem-loop structure.

60. The method of claim 42, wherein the heterologous nucleic acid sequence encodes a LoxP site.

61. The method of claim 42, wherein at least 10% of the RNA transcribed from the nucleic acid sequence is not spliced.

62. The method of claim 61, wherein at least 20% of the RNA transcribed from the nucleic acid sequence is not spliced.

63. The method of claim 62, wherein at least 50% of the RNA transcribed from the nucleic acid sequence is not spliced.

64. The method of claim 42, wherein at least 10% of the RNA transcribed from the nucleic acid molecule is spliced.

65. The method of claim 64, wherein at least 20% of the RNA transcribed from the nucleic acid sequence is spliced.

66. The method of claim 65, wherein at least 50% of the RNA transcribed from the nucleic acid sequence is spliced.

67. The method of claim 42, wherein the alternate form of the RNA molecule is translated in the cell to produce an alternate form of a protein.

68. The method of claim 67, wherein the protein is an antibody or an antigen binding portion thereof.

69. An isolated host cell comprising the nucleic acid sequence of claim 22, wherein (i) at least two different RNA transcripts are produced in the host cell by the nucleic acid sequence, and (ii) at least a first RNA transcript encodes a protein that is secreted by the host cell and at least a second RNA transcript encodes a membrane-bound protein.

70. An expression vector comprising the nucleic acid sequence of claim 22.

71. An isolated host cell comprising the expression vector of claim 70.

72. A protein generated by expression of the nucleic acid sequence of claim 22.

73. The protein of claim 72, wherein the protein is a chimeric protein.

74. The protein of claim 72, wherein the protein is a fusion protein.
